(19) United States
(12) Patent Application Publication
(10) Pub. No.: US 2018/0318381 A1
(43) Pub. Date: Nov. 8, 2018

(54) Methods of Treatment of Rhabdomyolysis

(71) Applicant: University of Pittsburgh - Of the Commonwealth System of Higher Education, Pittsburgh, PA (US)

(72) Inventors: Gerard Vockley, Pittsburgh, PA (US); Al-Walid A. Mohsen, Gibsonia, PA (US)

(73) Assignee: University of Pittsburgh- Of the Commonwealth System of Higher Education, Pittsburgh, PA (US)

(21) Appl. No.: 15/769,992

(22) PCT Filed: Oct. 21, 2016
(86) PCT No.: PCT/US16/58071

§ 371 Date: Apr. 20, 2018

Related U.S. Application Data
(60) Provisional application No. 62/245,309, filed on Oct. 23, 2015.

(51) Int. Cl. A61K 38/07; A61K 31/225; A61K 31/277; A61K 31/7072; A61P 29/00; A61P 3/00

(52) U.S. Cl. CPC A61K 38/07; A61K 31/225; A61P 3/00; A61K 31/7072; A61P 29/00; A61K 31/277

(57) ABSTRACT

Provided herein are methods of treating long-chain fatty acid disorders, conditions, such as rhabdomyolysis, associated with inflammation and/or long-chain fatty acid disorders, and inflammation associated with long-chain fatty acid disorders.

### CROSS REFERENCE TO RELATED APPLICATIONS

This application claims the benefit of U.S. Provisional Patent Application No. 62/245,309, filed Oct. 23, 2015, which is incorporated herein by reference in its entirety.

Inborn errors of fatty acid oxidation (FAO) have emerged as an increasing health problem. Now the most common group of disorders identified through expanded newborn screening, mandated in all 50 states in the US, they affect 2-3/1,000 babies born nationwide. Most importantly, in FAO disorders (FAOD's) the morbidity associated with symptomatic diagnosis can be reduced or eliminated if identified through screening. Unfortunately, a significant gap remains between our ability to treat them.

The Enzymes of β-Oxidation.

β-oxidation is the process through which fat is broken down for energy by a sequential cleavage of 2-carbon units from fatty acids. The complete process of mitochondrial β-oxidation involves activating fatty acids by esterification to coenzyme A, transporting the activated acyl-CoA moieties into the mitochondria, and sequentially removing 2 carbon acetyl-CoA units (See, FIG. 1).

These in turn are used as fuel for the tricarboxylic acid cycle or the production of ketone bodies (which can be exported to distant tissues such as the brain and utilized as an auxiliary fuel). The end result is the generation of reducing equivalents that are funneled into the electron transport chain and ultimately lead to the production of ATP. Short and medium chain CoA's can pass into the mitochondria independently, but long chain acyl-CoAs require active transport into mitochondria via carnitine products. CPT I is the overall rate-limiting step for β-oxidation. The long chain acylcarnitines are transported across to the inner mitochondrial membrane by the carnitine-acylcarnitine translocase (CACT), and then passed to CPT II which releases the carnitine and long chain acyl CoA into the mitochondrial matrix. Carnitine is itself transported intracellularly by the OCTN2 transporter protein encoded by the SLC22A5 gene. Once present in the mitochondrial matrix, acyl-CoAs of all chain lengths undergo β-oxidation, a four step series of enzymatic reactions. Each four step cycle results in the release of a two carbon acetyl-CoA (that can enter the tricarboxylic acid cycle or be converted to a ketone body), and a new acyl-CoA molecule that is two carbons shorter (see FIG. 1). The fatty acid undergoes successive cycles of β-oxidation until the process is completed. A number of the enzymes involved in the process are specific to the fatty acid's remaining chain length, such that unique enzymes are utilized in the oxidation of very long chain, long chain (for example, C₁₃₋₂₁), medium chain and short chain fatty acids. The successive steps of β-oxidation can be summarized as follows:

1. The first step in β-oxidation is the dehydrogenation of the beta carbon to create a double bond between the alpha and beta carbons creating a 2-enoyl-CoA. This reaction is catalyzed by a family of related enzymes, the acyl-CoA dehydrogenases (ACADs). Five different members of this family are active in β-oxidation: very long, long, medium, and short chain acyl-CoA dehydrogenases (VLCAD, LCAD, MCAD, and SCAD respectively) and ACAD9, that differ in their chain length specificity. These ACDs differ from most other dehydrogenases because they utilize electron transfer flavoprotein (ETF) as a final electron acceptor instead of NAD.

2. The 2-enoyl-CoA moieties generated by the ACADs are modified by a reductase adding water across the new double bond between the alpha and beta carbons. This generates a new hydroxyl group at the beta carbon, a 3-hydroxyacyl-CoA.

3. The 3-hydroxyacyl CoA undergoes another dehydrogenation using NAD⁺ at the β-carbon to create a 2-ketoacyl-CoA.

4. Finally, the 2-ketoacyl-CoA is cleaved by a thiolase to release the 2 carbon acetyl-CoA and completes one turn of the β-oxidation cycle.

The mechanism of steps 2-4 varies for substrates of differing chain length. For longer chain acyl-CoA substrates, all 3 enzymes [2-enoyl-CoA hydratase, 3-hydroxy-acyl-CoA dehydrogenase (LCHAD), and 3-ketoacyl-CoA thiolase (LKAT)] are contained within the mitochondrial trifunctional protein (TFP). For medium or short chain fatty acids, different enzymes are involved including a short/medium chain 3-hydroxyacyl-CoA dehydrogenase (S/MCHAD), a short chain enoyl-CoA hydratase (also called crotonase), and distinct medium and short chain 3-ketoacyl-CoA thiolases (MKAT and SKAT). In addition, if the fatty acyl-CoA is unsaturated, additional enzymes are required, including a 2,4-dienoyl-CoA reductase and a Δ³, Δ²-enoyl-CoA isomerase.

Disorders of β-Oxidation:

The clinical disorders of β-oxidation are highly pleiotropic. The cardinal sign shared by all FAODs include recurrent hypoglycemia developing into Reye-like syndrome induced by fasting or physiologic stress. Additional clinical presentations can range from overwhelming neonatal acidosis to adult onset recurrent rhabdomyolysis or exercise intolerance, or affected individuals can also remain asymptomatic for life. Some broad generalizations can be made; long chain FAODs frequently involve muscular symptoms, including cardiomyopathy, myopathy, and rhabdomyolysis in addition to hypoglycemia. Less specific symptoms including failure to thrive and hypotonia may be the only clues to shorter chain defects. The clinical variability is great enough to preclude a definitive diagnosis on the basis of clinical symptoms alone, and genotype is in general a poor predictor of phenotype. Deficiencies of several steps of LC-FAO have been described:

CPTIa/CACT/CPTII. These disorders can present quite similarly though with a broad clinical spectrum. They generally characterized by episodic hypoketotic hypoglycemia, often with hyperammonemia. Severe cases can begin in infancy and be accompanied by multi-organ system failure. CPTII deficiency is the most common of this group and has a characteristic sub group with adolescent onset phenotype with recurrent rhabdomyolyis.

VLCAD. All patients can present with hypoglycemia and Reye-like syndrome with hypoketotic hypoglycemia, hyperammonemia, and hepatocellular failure upon fasting. Severely affected patients with early onset cardiomyopathy and severe ventricular cardiac arrhythmias. Adolescents or adults can develop exercise intolerance, skeletal myopathy and episodes of rhabdomyolysis. Expanded newborn screening has identified a moderately high incidence of VLCAD deficiency in the US (1/31,500). At present there is a concerning shortage of outcomes studies and evidence-based treatment protocols.

ACAD9. This is both the most recently identified enzyme involved in fatty acid oxidation, and the most recent disorder. Deficiency of ACAD9 results in 2 phenotypes. ACAD9 deficiency was first shown to present with episodic hepatocellular dysfunction and can proceed to liver failure, Reye syndrome, and death if untreated. It was subsequently identified in isolated respiratory chain complex I deficiency leading to cardiomyopathy+/− systemic involvement. This surprising finding is explained by a non-enzymatic moonlighting function of the ACAD9 protein as complex I assembly factor. ACAD9 is now recognized as the most common cause of isolated complex I deficiency.

MADD (multiple acyl-CoA dehydrogenase deficiency). Abnormalities of ETF or ETF:ubiquinone oxidoreductase (ETF dehydrogenase) deficiency lead to an in vivo deficiency of all of the dehydrogenases which utilize ETF as an electron acceptor. Because of the presence of glutaric acid in the urine of some patients, this disorder is frequently referred to as glutaric aciduria type 11 (GA II) to distinguish it from a primary deficiency of glutaryl-CoA dehydrogenase (GA I). Clinical manifestations of MADD are extremely heterogeneous. A severe and fatal neonatal form presents with severe hypotonia, dysmorphic features, and cystic kidneys. Structural brain abnormalities are common including agenesis of the cerebellar vermis, hypoplastic temporal lobes, and focal dysplasia of cerebral cortex. Neuronal migration abnormalities may be present. A milder childhood form usually in infants with metabolic acidosis, hypoglycemia, hyperammonemia, liver dysfunction, bone marrow depression, and muscle weakness. Complications include severe cardiomyopathy and leukodystrophy. Older patients in childhood up to young adults present with lipid storage myopathy and are often riboflavin responsive.

LCHAD/TFP. Patients with a deficiency of LCHAD tend to fall into two general subclasses. Patients present with hypoglycemia, liver dysfunction and Reye-like syndrome upon fasting. They can also exhibit cardiomyopathy, and myopathy including recurrent rhabdomyolysis. Symptoms particular to this disorder include: cholestatic liver disease, peripheral neuropathy and pigmentary retinopathy. There sometimes is a history of maternal illness during pregnancy of the affected child. Milder cases with adolescent onset of recurrent rhabdomyolysis have also been reported.

Newborn screening. Screening for many disorders of j3-oxidation using tandem mass spectrometry of dried blood spots collected prior to discharge from the nursery has been implemented in most states and is being evaluated in a number of others. Pre-symptomatic identification of these individuals can prevent catastrophic events, especially sudden death. Savings in medical expenses related to newborn screening must also be considered as a benefit of such screening. The current standard of care for LC-FAOD is dietary management. Treatment may vary according to the specific disorder and the severity of the underlying enzyme deficiency, but may include a low fat, high carbohydrate diet, the avoidance of fasting, and supplementation with carnitine and/or medium even chain triglyceride (MCT) oil, along with aggressive treatment of co-morbid illness. The effectiveness of therapies has largely been reported on a case-by-case basis with little information available from well-controlled clinical studies. A retrospective analysis of 187 clinically diagnosed patients with LC-FAOD concluded that mortality rates have not changed overall for patients during the past 30 years, even with an evolving standard of care, and overall there is greater than 50% mortality in each decade, with some indications having mortality in the 60-95% range during their period of review (Baruteau J, et al. (2013). Clinical and biological features at diagnosis in mitochondrial fatty acid beta-oxidation defects: a French pediatric study of 187 patients. J Inherit Metab Dis. 36: 795-803). However, newborn screening and early treatment may reduce mortality and improve outcomes, although long-term experience has not yet been published. Despite diagnosis and treatment from the newborn period, one study demonstrated that FAOD subjects still had a major decompensation rate of ˜25% in the two years after birth. Thus, patients require better treatment options, particularly to prevent the major decompensation events that lead to hospitalization and major morbidity and mortality.

Treatment of LC-FAODs: Treatment of LC-FAODs has largely been symptomatic. We have recently been involved in the development of triheptanonin, an anapleurotic agent that we hypothesize corrects a secondary imbalance in the TCA cycle in patients with LC-FOADs. In a retrospective study of 20 subjects treated with triheptanoin followed over 15 years, 320 hospitalizations were recorded from birth to the end date of study. The mean hospitalization days/year decreased significantly by 67% during the period after triheptanoin initiation (n=15; 5.76 vs 17.55 vs; P=0.0242) and a trend toward a 35% lower hospitalization event rate was observed in the period after triheptanoin initiation compared with the before-treatment period (n=16 subjects >6 months of age; 1.26 vs 1.94; P=0.1126). The hypoglycemia event rate per year in 9 subjects with hypoglycemia problems declined significantly by 96% (0.04 vs 0.92; P=0.0091) and related hospitalization days/year were also significantly reduced (n=9; 0.18 vs 8.42; P=0.0257). The rhabdomyolysis hospital event rate in 11 affected subjects was similar before and after treatment but the number of hospitalization days/year trended lower in the period after triheptanoin initiation (n=9; 2.36 vs 5.94; P=0.1224) and peak CK levels trended toward a 68% decrease from 85,855 to 27,597 units in 7 subjects with reported peak CK values before and after treatment (P=0.1279). Thus, while triheptanoin was effective in preventing hypoglycemia, rhabdomyolysis remains a major problem.

Fibrates, acting as agonist of peroxisome proliferator-activated receptors (PPARs), have been shown to stimulate FAO in some FAO-deficient cells. Bezafibrate or fenofibric acid in the culture medium induced a dose-dependent increase in palmitate oxidation capacities in cells from patients with the myopathic form of VLCAD deficiency, but not in cells from severely affected patients, and in cells from CPT2 deficient patients. Real-time PCR studies indicated that bezafibrate potentially stimulated gene expression of other enzymes in the beta-oxidation pathway. Small clinical trials showed some benefit to fibrate treatment of patients with GA2 but not CPT2. Thus, transcriptional activators as therapy for FAODs remains attractive.

There is therefore a need for drug products that are useful in treatment of fatty acid oxidation disorders, such as rhabdomyolysis caused by defects in fatty acid oxidation disorders.

### SUMMARY

Accordingly, a method of treating a long-chain fatty acid oxidation disorder is provided. In another aspect, a method of treating inflammation associated with a long-chain fatty acid oxidase disorder is provided. In a further aspect, a method of treating inflammation associated with rhabdomyolysis associated with a long-chain fatty acid oxidation disorder is provided. The methods comprise administering to a patient a mitochondrial antiinflammatory composition, that, for example, up-regulates fatty acid metabolism in a patient or stabilizes mitochondrial structure, in an amount effective to improve fatty acid oxidation function, improve mitochondrial respiratory chain function, increase mitochondrial stability, treat rhabdomyolysis, and/or decrease inflammation in a patient. Examples of mitochondrial antiinflammatory composition include Bendavia™, and analogs thereof, RTA-408 and analogs thereof, and uridine triacetate.

### BRIEF DESCRIPTION OF THE DRAWINGS

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the invention.

FIG. 1: Fatty acid oxidation and its relationship to other energy cycles. Metabolism of triheptanoin is shown.

FIG. 2 provides exemplary amino acid sequences for various Bendavia™ analogs.

FIG. 3: Patients with VLCAD deficiency show an atypical inflammatory pattern in blood consistent with macrophage activation (top). Macrophages indeed are in an activated state, even when patients are well (bottom).

FIG. 4: mRNA was isolated from muscle from wild type and VLCAD deficient animals and concentrations were compared by expression array analysis.

FIG. 5: Genes involved in interferon-mediated inflammation are elevated in VLCAD deficient animals.

FIG. 6: Genes involved in macrophage-mediated inflammation are elevated in VLCAD deficient animals.

FIG. 7: Genes involved in T cell-mediated inflammation are elevated in VLCAD deficient animals.

FIG. 8: Genes involved in T cell-mediated inflammation are elevated in VLCAD deficient animals.

FIG. 9 is a graph showing cytokine analysis of serum from wild type and VLCAD deficient mice.

FIG. 10 provides a Luminex profile of cytokines from (n=16) VLCAD patients.

FIG. 11 is a graph showing mean fluorescence intensities of cytokines in CD16+/− cells of a VLCAD patent with recurring rhabdomyolysis.

FIG. 12 is a graph showing measurement of whole cell oxygen consumption in VLCAD and LCHAD deficient cell lines.

FIG. 13 is a summary of the cellular experiments illustrating that overall oxygen consumption is reduced in VLCAD deficient cell lines, whereas oxygen consumption is increased in LCHAD deficient cell lines.

FIG. 14 is a table providing hospitalization data for hypoglycemia and rhabdomyolysis.

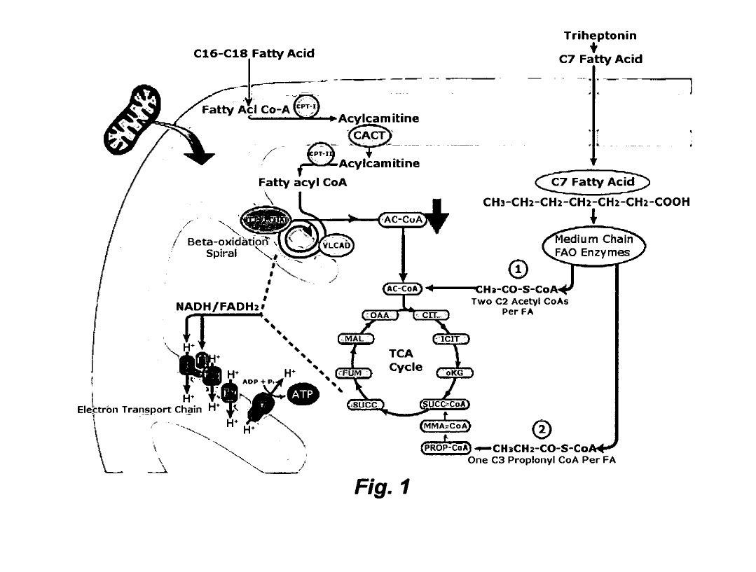

### DETAILED DESCRIPTION

As used herein, the singular form of “a”, “an”, and “the” include plural referents unless the context clearly dictates otherwise.

It is to be understood that the invention can assume various alternative variations and stage sequences, except where expressly specified to the contrary. It is also to be understood that the specific compounds, compositions, and processes illustrated in the attached drawings, and described in the following specification, are examples. Hence, specific dimensions and other physical characteristics related to the embodiments disclosed herein are not to be considered as limiting.

Unless indicated to the contrary, the numerical parameters set forth in the following specification and attached claims are approximations that can vary depending upon the desired properties sought to be obtained by the present disclosure.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

A “patient” is any member of the animal kingdom, for example mammals or humans. “Patient” does not imply or require any doctor-patient or veterinarian-patient relationship.

“Treatment” of a disease or condition in a patient means improvement of one or more testable, clinically-relevant markers, such as symptoms, physiological response, gene expression, analyte production or prevalence, or any other assessable effect. “Improvement” means that the value obtained for the clinically relevant marker objectively changes toward a normal or more medically- or veterinarially-desirable value indicative of improvement in health of the patient. Non-limiting examples of markers for FAODs include hypoglycemia, Reye-like syndrome induced by fasting or physiologic stress, neonatal acidosis, rhabdomyolysis, adult onset recurrent rhabdomyolysis, and/or exercise intolerance.

By “expression,” it is meant the overall process by which a gene is used to produce a gene product, such as an RNA or protein, and can be determined by, for example, mRNA production or by functional assays for the gene product. Lower expression means reducing the amount of a gene product as compared to the gene when its expression is not lowered. “Downregulation” of expression or function is a lowering of expression or function to any relevant degree, e.g., to a statistically significant degree. In contrast, “upregulation” of expression or function is an increase of expression or function to any relevant degree, e.g., to a statististically significant degree.

Long-chain fatty acid oxidation disorders (LC-FAODs) represent a group of autosomal recessive inborn errors of metabolism with an estimated prevalence of ˜1:17,000 in the US, based on newborn screening data. LC-FAOD are caused by defects in nuclear genes that encode six mitochondrial enzymes involved in the oxidation of long chain fats for energy during times of fasting and physiologic stress. The genes and their associated diseases include carnitine palmitoyl transferase 1 (CPT-I), carnitine palmitoyl transferase 2 (CPT-II), carnitine/acylcarnitine translocase (CACT), very long-chain acyl-CoA dehydrogenase (VLCAD), long-chain 3-hydroxyacyl-CoA dehydrogenase (LCHAD), and mitochondrial trifunctional protein (TFP) deficiencies. As a result of the enzymatic block, partial or incomplete oxidation of fatty acids occurs, leading to accumulation of high concentrations of potentially toxic fatty acid intermediates and an energy deficit state in many organ systems. Management of LC-FAODs includes diligent avoidance of fasting combined with the use of low fat/high carbohydrate diets, carnitine supplementation in some cases, medium chain triglyceride (MCT) oil supplementation. A comprehensive clinical survey over 30 years of experience and 187 cases at one center suggests LC-FAODs as a group have a mortality rate of 50% or higher when diagnosed symptomatically, despite increased awareness of the disorders and management over the last 2 decades (Baruteau, J., et al., Clinical and biological features at diagnosis in mitochondrial fatty acid beta-oxidation defects: a French pediatric study of 187 patients. J Inherit. Metab Dis. 2012). Newborn screening and early treatment have reduced mortality, but carefully followed cohorts indicate major medical events continue to occur despite newborn screening diagnosis and management.

Patients with LC-FAOD present at any age with acute crises of energy metabolism and severe energy deficiency, even with treatment compliance rates of >80%. The main presentations are characterized by involvement of the liver, skeletal muscle, or heart associated with hypoglycemia/liver dysfunction early in life, muscle weakness/rhabdomyolysis later in life, and episodic cardiomyopathy with or without arrhythmias at any age. The pattern and severity of organ involvement are generally not predictable based on the inherited defect.

Traditional care for LC-FAOD includes supplementation with MCT oil, containing predominantly eight and ten carbon triglycerides. Since the long-chain fatty acid oxidation enzymes are not required to metabolize MCT, they can bypass the primary metabolic defect providing acetyl-CoA for the Krebs cycle. However, the Krebs cycle requires both even and odd chain intermediates to function, and thus may become secondarily impaired with this therapy, as observed directly in the heart of a murine FAOD model during heart failure. The energy deficiency is accentuated by the need for aggressive gluconeogenesis with severe hypoglycemia and depletion of glycogen, which has the potential to further drain TCA cycle intermediates to feed the gluconeogenesis pathway.

The basic science of fatty acid oxidation and the pathophysiology of fatty acid oxidation disorders (FAODs) has led to the identification and partial purification of a multifunctional fatty acid oxidation complex that contains all of the enzymes involved in FAOD. The complex interacts functionally and physically with the mitochondrial respiratory chain, establishing a new paradigm for understanding the effects of mutations in these pathways on energy metabolism. Long chain FAODs induce an atypical inflammatory response that is thought to play a role in the development of recurrent rhabdomyolysis. To date, no specific therapies are approved for long chain FAODs. Triheptanoin, an investigational drug that better addresses metabolic imbalances induced by FAODs, appears to improve some symptoms, but is not effective in preventing the recurrent rhabdomyolysis that is the major cause of morbidity in older children and adults with these disorders. Bezafibrate, an activator of FAOD gene transcription, has shown some benefit in improving in vitro FAO, but has not been extensively tested in patients.

Small molecular weight compounds are described herein that alter gene transcription to treat rhabdomyolysis in patients with FAODs, for example in those with rhabdomyolysis of unknown cause. The compounds described herein have the biological effect of reducing mitochondrial-induced inflammatory signals, and therefore are referred to herein as “mitochondrial-antiinflammatory compound(s)” or “mitochondrial-antiinflammatory composition(s)” because their antiinflammatory activity is directed to an aspect of mitochondrial function or structure. Mitochondria-directed antiinflammatory activity can be accomplished by a variety of mechanisms related to mitochondria function. First, increasing mitochondrial energy metabolism reduces inflammation since energy deficiency leads to mitochondrial instability, which in turn induces apoptosis and inflammation. compounds or compositions that upregulate components of energy metabolism (fatty acid oxidation or the respiratory chain) work through this mechanism (e.g., RTA-408). Other compounds or compositions work through direct stabilization of the mitochondrial membrane (e.g., Bendavia) or the ionic composition of the mitochondria (e.g., uridine triacetate). Other compounds influence the expression of genes involved in regulating mitochondrial induced inflammation as well as the enzymes themselves.

As a class, these compounds can be described as, and/or have mitochondrial-antiinflammatory activity, anti-oxidation effect and/or up-regulate (increases expression) of fatty acid metabolism genes, e.g., nuclear-encoded mitochondrial genes. With respect to nuclear-encoded mitochondrial genes, compounds that alter the transcription of nuclear-encoded mitochondrial genes involved in fatty acid oxidation. Specifically, because these compounds act as global transcriptional activators of the genes involved in energy metabolism, treatment using these compounds will improve in vitro FAO in cells derived from patients with disorders in this pathway. Further, they will decrease the atypical inflammatory response seen in mouse models of FAODs. As such, “mitochondrial-antiinflammatory compounds” include compounds that increase mitochondrial energy metabolism and/or stabilize the mitochondrial membrane. Examples of such mitochondrial-antiinflammatory compounds include Bendavia™ (Stealth Pharmaceuticals) and RTA408 (Reata Pharmaceuticals).

According to one aspect, a method is provided for treatment of a long-chain fatty acid oxidation disorder. According to another aspect, a method is provided for treatment of inflammation associated with rhabdomyolysis associated with a long-chain fatty acid oxidation disorder. According to another aspect, a method is provided for treatment of inflammation associated with a long-chain fatty acid oxidase disorder. The methods comprise administering to a patient a mitochondrial antiinflammatory composition, that optionally up-regulates fatty acid metabolism in a patient or stabilizes mitochondrial structure, in an amount effective to improve fatty acid oxidation function (that is, to bring one or more indicators of deficient fatty acid metabolism, such as expression of one or more genes affecting fatty acid metabolism closer to normal expression levels), improve mitochondrial respiratory chain function, increase mitochondrial stability, treat rhabdomyolysis, and/or decrease inflammation in a patient, such as rhabdomyolysis or inflammation associated with a long-chain fatty acid oxidase disorder. In one aspect, the composition comprises Bendavia, or an analog, or a pharmaceutically acceptable salt or ester thereof.

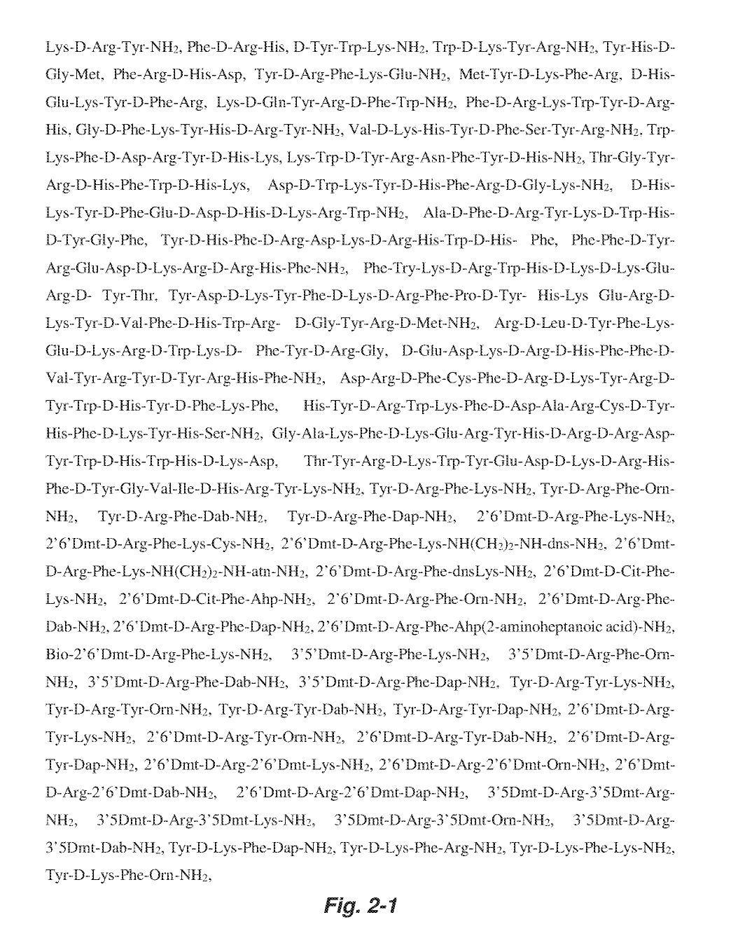

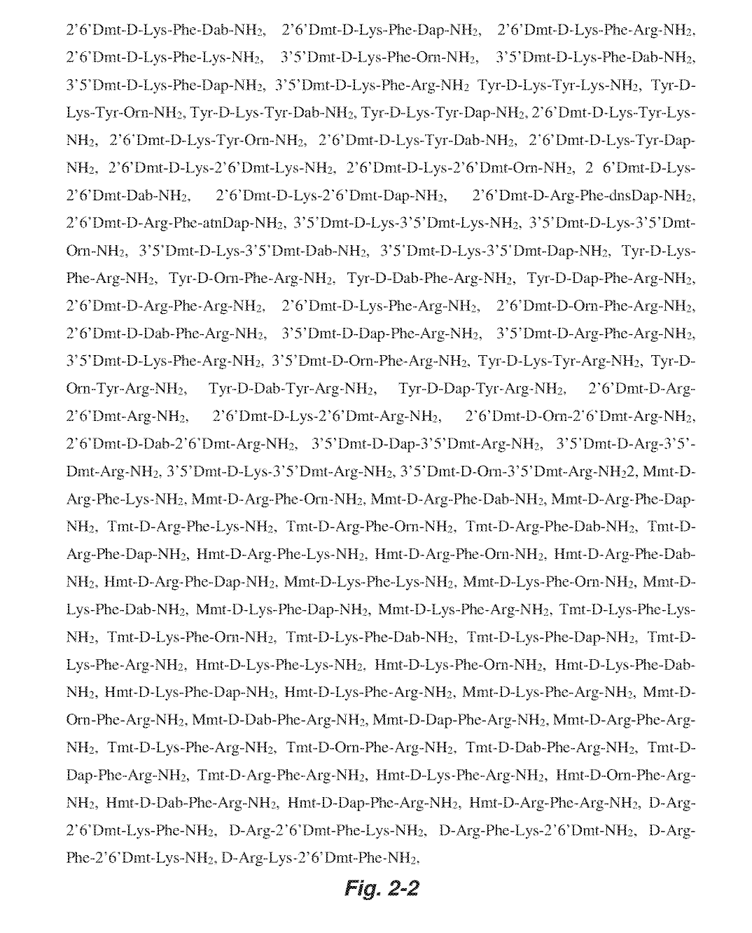

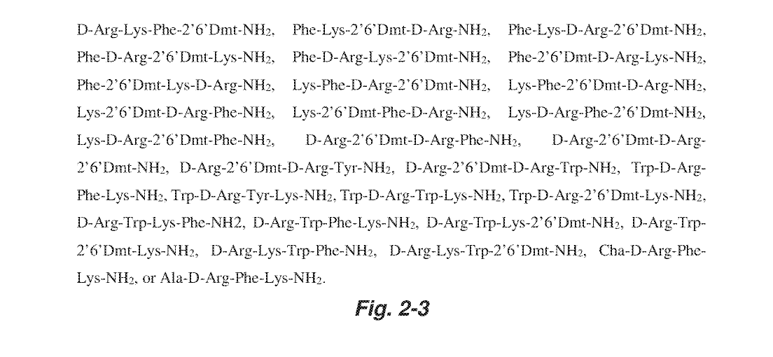

Bendavia™ (also, elamipretide or MTP-131, Stealth Pharmaceuticals) is a tetrapeptide derivative (D-Arg-2′6′-Dimethyltyrosine-Lys-Phe-NH₂) that binds the hydrophilic head of oxidized cardiolipin moieties in the mitochondrial membrane and restores its molecular structure to normal, reducing apoptosis in the setting of hypoxia due to ischemia (Dai W, et al., “Bendavia, a mitochondria-targeting peptide, improves postinfarction cardiac function, prevents adverse left ventricular remodeling, and restores mitochondria-related gene expression in rats”, J. Cardiovasc Pharmachol., 2014, 64: 543-553; Brown D A, et al., “Reduction of early reperfusion injury with the mitochondria-targeting peptide bendavia”, J. Cardiovasc Pharmachol Ther., 2014, 19: 121-132; Chakrabarti A K, et al., “Rationale and design of the EMBRACE STEMI study: a phase 2a, randomized, double-blind, placebo-controlled trial to evaluate the safety, tolerability and efficacy of intravenous Bendavia on reperfusion injury in patients treated with standard therapy including primary percutaneous coronary intervention and stenting for ST-segment elevation myocardial infarction”, Am Heart J. 2013 April, 165(4):509-514; and Kloner R A, et al., “Reduction of ischemia/reperfusion injury with bendavia, a mitochondria-targeting cytoprotective Peptide”, J Am Heart Assoc. 2012 June; 1(3):e001644). It also functions as a modifying enzyme in cardiolipin remodeling (Taylor W A, et al., “Human trifunctional protein alpha links cardiolipin remodeling to Beta-oxidation”, PLoS One, 2012, 7: e48628). Bendavia™ up-regulates expression of nuclear-encoded mitochondrial genes, it reduces cardiomyocyte apoptosis post ischemia, and decreases amyloid B induced mitochondrial abnormalities. Bendavia™ additionally improves skeletal muscle function by affecting flux of the electron transport chain, increasing ATP synthesis, and decreasing mitochondrial ROS (see FIG. 2 below).

Bendavia™ (D-Arg-2′6′-Dimethyltyrosine-Lys-Phe-NH₂) has the structure:

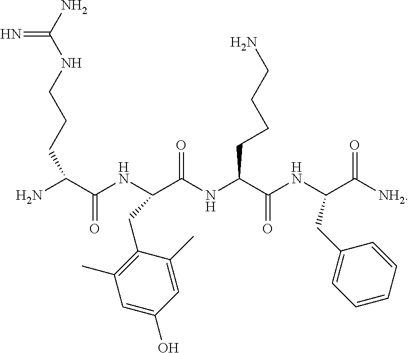 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

Bendavia™, and analogs thereof are described in United States Patent Application Publication Nos. 20110082084 and 20140100166, which are incorporated herein by reference for the compositions described therein. U.S. Pat. Nos. 8,404,646 and 9,150,614 describe therapeutic uses for Bendavia^{T}, and related aromatic cationic peptides analogs thereof, including, for example and without limitation aromatic-cationic peptides having an amino acid sequence shown in FIG. 2 (see, e.g., U.S. Pat. No. 8,404,646).

RTA408 (N-(2-cyano-3,12-dioxo-28-noroleana-1,9(11)-dien-17-yl)-2,2-difluoro-propanamide) is a compound having the structure:

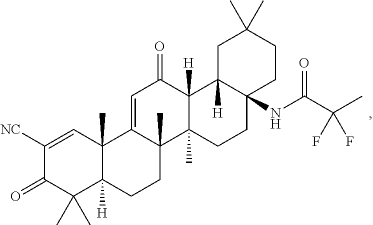

(Reata Pharmaceuticals; also known as TX63415, FP-190, ABT-RTA408, and A-1402484.0), and is a member of a class of semi-synthetic triterpenoids discovered through a medicinal chemistry effort to optimize the potency of these compounds to inhibit the induction of nitric oxide (NO) in primary mouse macrophages treated with interferon-gamma (Uruno A, et al., “The Keapl-Nrf2 system prevents onset of diabetes mellitus”, Mol Cell Biol., 2013, 33: 2996-3010; Holmstrom K M, el al., “Nrf2 impacts cellular bioenergetics by controlling substrate availability for mitochondrial respiration”, Biol Open. 2013, 2: 761-770; and Ludtmann M H, et al., “Nrf2 affects the efficiency of mitochondrial fatty acid oxidation”, Biochem J., 2014, 457: 415-424). Subsequent mechanistic studies have revealed that RTA408 and related semi-synthetic triterpenoids are potent activators of the Nrf2 promotor (nuclear factor erythroid-derived 2-related factor 2) and inhibitors of NF-κB (nuclear factor kappa-light-chain-enhancer of activated B-cells) and thus, induce an antioxidative and antiinflammatory phenotype. This compound increases the expression of FAO genes, similar to bezafibrate (see, e.g., Djouadi F, et al., “Bezafibrate increases very-long-chain acyl-CoA dehydrogenase protein and mRNA expression in deficient fibroblasts and is a potential therapy for fatty acid oxidation disorders”, Hum Mol Genet. 2005 Sep. 15; 14(18):2695-703; Bonnefont J P, et al., “Bezafibrate for an inform mitochondrial beta-oxidation defect”, N. Engl. J. Med., 2009, 360: 838-840; Yamaguchi S, et al., “Bezafibrate can be a new treatment option for mitochondrial fatty acid oxidation disorders: evaluation by in vitro probe acylcarnitine assay”, Mol. Genet. Metab., 2012, 107: 87-91; Omgreen M C, et al., “Bezafibrate in skeletal muscle fatty acid oxidation disorders: A randomized clinical trial”, Neurology, 2014, 82: 607-613; Djouadi F, et al., “Correction of fatty acid oxidation in carnitine palmitoyl transferase 2-deficient cultured skin fibroblasts by bezafibrate”, Pediatr Res. 2003 October; 54(4):446-51; Bonnefont J P, et al., “Long-term follow-up of bezafibrate treatment in patients with the myopathic form of carnitine palmitoyltransferase 2 deficiency”, Clin Pharmacol Ther. 2010 July; 88(1):101-8; Gobin-Limballe S, et al., “Genetic basis for correction of very-long-chain acyl-coenzyme A dehydrogenase deficiency by bezafibrate in patient fibroblasts: toward a genotype-based therapy”, Am J Hum Genet. 2007 December; 81(6):1133-43; Li H, et al., “Effect of heat stress and bezafibrate on mitochondrial beta-oxidation: comparison between cultured cells from normal and mitochondrial fatty acid oxidation disorder children using in vitro probe acylcarnitine profiling assay”, Brain Dev. 2010 May; 32(5):362-70; Dillon L M, et al. “Long-term bezafibrate treatment improves skin and spleen phenotypes of the mtDNA mutator mouse”, PLoS One. 2012; 7(9):e44335; and Engelen M, et al., “Bezafibrate lowers very long-chain fatty acids in X-linked adrenoleukodystrophy fibroblasts by inhibiting fatty acid elongation”, J Inherit Metab Dis. 2012 November; 35(6): 113745). However, it also reduces the transcription of a specific class of inflammatory proteins that are increased in patients with mitochondrial ETC (electron transport chain) deficiency. RTA408 has been shown to improve antioxidant gene expression in response to oxidative stress in Friedrich's ataxia cells. A compound related to RTA408, 2-cyano-3,12-dioxooleana-1,9-dien-28-oic acid-ethyl amide (CDDO-EA) has been show to improve survival in an amyotrophic lateral sclerosis mouse model (See, Neymotin A, et al. (2011). Neuroprotective effect of Nrf2/ARE activators, CDDO ethylamide and CDDO trifluoroethylamide, in a mouse model of amyotrophic lateral sclerosis. Free Radic Biol Med. 51: 88-96).

United States Patent Publication Nos. US20130324599 and U.S. Pat. No. 8,993,640 (both of which are incorporated herein by reference for their disclosure of the triterpenoid RTA408, and analogs thereof) disclose RTA408 and related semi-synthetic triterpenoids, such as RTA 401, that are potentially useful in the methods described herein, and methods of synthesis thereof, and that are referred to herein as “RTA408 analogs.”

Triheptanoin (1,3-di(heptanoyloxy)propan-2-yl heptanoate) has the structure:

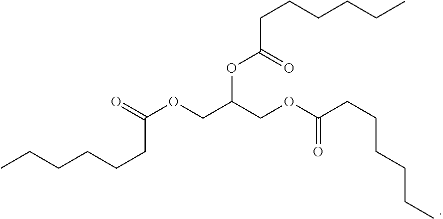 [SMILES: CCCCCCC(=O)OCC(COC(=O)CCCCCC)OC(=O)CCCCCC]

It is initially catabolized in the gut to free heptanoate that can diffuse across membranes to enter cells. Heptanoyl-CoA is then metabolized by medium chain fatty acid oxidation enzymes to acetyl- and propionyl-CoA, as well as 4- and 5-carbon ketone bodies. Propionyl-CoA is an anaplerotic molecule that restores the balance of the Krebs cycle intermediates pool via conversion to succinyl-CoA (Brunengraber, H., et al. Anaplerotic molecules: current and future. J. Inherit. Metab Dis. 2006; 29:327-331). The restoration of Krebs cycle function improves flow of electrons to the mitochondrial respiratory chain recovering ATP production (Kinman, R. P., et al. Parenteral and enteral metabolism of anaplerotic triheptanoin in normal rats. Am. J. Physiol Endocrinol. Metab. 2006; 291:E860-E866).

Uridine triacetate (marketed as Vistogard®) is a pyrimidine analog, and is currently used as a cancer therapeutic. Following oral administration, uridine triacetate is deacetylated by nonspecific esterases present throughout the body, yielding uridine in the circulation. Uridine competitively inhibits cell damage and cell death caused by fluorouracil (5-FU). 5-FU interferes with nucleic acid metabolism (DNA and RNA) in normal and cancer cells. Cells anabolize 5-FU to the cytotoxic intermediates FdUMP (5-fluoro-2′-deoxyuridine-5′-monophosphate) and FUTP (5-fluorouridine triphosphate). FdUMP inhibits synthesis of thymidine, which is required for DNA replication and repair. Uridine is not found in DNA. The incorporation of FUTP into RNA is a major source of 5-FU cytotoxicity. Excess circulating uridine derived from uridine triacetate is converted into UTP (uridine triphosphate), which competes with FUTP for incorporation into RNA. Uridine triacetate inhibits incorporation of FUTP into RNA, a major source of 5-FU toxicity.

Uridine triacetate has been shown to regulate mitochondrial ATP-sensitive potassium channels by preventing ATP depletion, Ca⁺⁺ overload, and ROS production, and also by regulating mitochondrial volume and pH. Uridine triacetate is able to activate mitochondrial ATP-sensitive K+ channels, which increases ATP synthesis rate in hypoxic tissues. Evidence also suggests that this drug decreases inflammatory signaling. Treatment of mice with the mitochondrial toxin azide leads to rapid death. Supplementation of animals with PN401 (triacetyluridine) at the same time as azide, leads to improved survival of the animals.

Pharmaceutically acceptable salts of any of the compounds described herein also may be used in the methods described herein. Pharmaceutically acceptable salt forms of the compounds described herein may be prepared by conventional methods known in the pharmaceutical arts, and include as a class veterinarially acceptable salts. For example and without limitation, where a compound comprises a carboxylic acid group, a suitable salt thereof may be formed by reacting the compound with an appropriate base to provide the corresponding base addition salt. Non-limiting examples include: alkali metal hydroxides, such as potassium hydroxide, sodium hydroxide and lithium hydroxide; alkaline earth metal hydroxides, such as barium hydroxide and calcium hydroxide; alkali metal alkoxides, such as potassium ethanolate and sodium propanolate; and various organic bases such as piperidine, diethanolamine, and N-methylglutamine.

Acid and base addition salts may be prepared by contacting the free base form with a sufficient amount of a desired acid or base to produce the salt in a manner known in the art. The free base may be regenerated by contacting the salt form with a base or acid (depending on the nature of the salt) and isolating the free base. The free base forms differ from their respective salt forms somewhat in certain physical properties such as solubility in polar solvents, but otherwise the salts are equivalent to their respective free base forms for purposes described herein.

Compounds comprising basic nitrogen-containing groups may be quaternized with such agents as C₁₋₄ alkyl halides, such as methyl, ethyl, iso-propyl and tert-butyl chlorides, bromides and iodides; C₁₋₄ alkyl sulfate such as dimethyl, diethyl and diamyl sulfates; C₁₀₋₁₈ alkyl halides, such as decyl, dodecyl, lauryl, myristyl and stearyl chlorides, bromides and iodides; and aryl-C₁₋₄ alkyl halides, such as benzyl chloride and phenethyl bromide. Such salts permit the preparation of both water-soluble and oil-soluble compounds.

Non-limiting examples of pharmaceutically-acceptable base salts include: aluminum, ammonium, calcium, copper, ferric, ferrous, lithium, magnesium, manganic, manganous, potassium, sodium, and zinc salts. Salts derived from pharmaceutically acceptable organic non-toxic bases include, without limitation: salts of primary, secondary, and tertiary amines, substituted amines including naturally occurring substituted amines, cyclic amines, and basic ion exchange resins, such as arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris-(hydroxymethyl)-methylamine (tromethamine).

Acid addition salts may be prepared by treating a compound with pharmaceutically acceptable organic and inorganic acids, including, without limitation: hydrohalides, such as hydrochloride, hydrobromide, hydroiodide; other mineral acids and their corresponding salts such as sulfates, nitrates, and phosphates; alkyl- and mono-arylsulfonates, such as ethanesulfonate, toluenesulfonate, and benzenesulfonate; and other organic acids and their corresponding salts, such as acetate, tartrate, maleate, succinate, citrate, benzoate, salicylate, and ascorbate.

Non-limiting examples of pharmaceutically-acceptable acid salts include: acetate, adipate, alginate, arginate, aspartate, benzoate, besylate (benzenesulfonate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate, galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate, and phthalate.

Multiple salts forms are also considered to be pharmaceutically-acceptable salts. Common, non-limiting examples of multiple salt forms include: bitartrate, diacetate, difumarate, dimeglumine, diphosphate, disodium, and trihydrochloride.

As such, “pharmaceutically acceptable salt” as used herein is intended to mean an active ingredient (drug) comprising a salt form of any compound as described herein. The salt form preferably confers to the improved and/or desirable pharmacokinetic/pharmodynamic properties of the compounds described herein.

“Pharmaceutically acceptable esters” includes those derived from compounds described herein that are modified to include a carboxyl group. An in vivo hydrolysable ester is an ester, which is hydrolysed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C₁₋₄ alkyl, or C₁₋₄ alkoxy) or amino); sulphonate esters, such as alkyl- or aralkylsulphonyl (for example, methanesulphonyl); or amino acid esters (for example, L-valyl or L-isoleucyl). A “pharmaceutically acceptable ester” also includes inorganic esters such as mono-, di-, or triphosphate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and valyl.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art.

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group and/or a phosphonate ester or phosphonate amide group. In particular examples, a prodrug is a lower alkyl phosphonate ester, such as an isopropyl phosphonate ester.

As used herein, unless indicated otherwise, for instance in a structure, all compounds and/or structures described herein comprise all possible stereoisomers, individually or mixtures thereof. The compound and/or structure may be an enantiopure preparation consisting essentially of an (−) or (+) enantiomer of the compound, or may be a mixture of enantiomers in either equal (racemic) or unequal proportions.

In use, any compound described herein, including pharmaceutically acceptable salts thereof, may be admixed with any pharmaceutically acceptable carrier or carriers, such as water, saline, physiological salt solutions, Ringer's solution or any other carrier customarily used for administration of drugs to the subject in question (see, generally, Troy, D B, Editor, Remington: The Science and Practice of Pharmacy, 21st Ed., Lippincott Williams & Wilkins (2005), pp. 745-849 for descriptions of various compositions, solutions, and dosage forms useful for administration of the described compounds, as well as methods of making such compositions, solutions, and dosage forms).

According to one non-limiting example, the compounds described herein are formulated into a composition, such as a drug product with one or more additional pharmaceutically acceptable excipients, e.g., vehicles or diluents for oral, intravenous or subcutaneous administration. The composition can be formulated in a classical manner using solid or liquid vehicles, diluents and additives appropriate to the desired mode of administration. Orally, the compounds can be administered in the form of tablets, capsules, granules, powders and the like. The compositions optionally comprise one or more additional active agents, as are broadly known in the pharmaceutical, medicinal, veterinary or biological arts.

The compounds described herein may be administered in any manner that is effective to treat a fatty acid oxidation defect, such as rhabdomyolysis, thereby improving one or more symptoms, pathologies, sequelae, effects, etc. of rhabdomyolysis. Rhabdomyolysis is a breakdown of muscle tissue, resulting in the release of muscle fiber contents into the blood, resulting in harm to the kidney. Rhabdomyolysis can result from various injuries to muscle tissue, including traumatic (e.g., crushing) injury, loss of blood supply, metabolic effects, hyperthermia drug use, such as a side-effect of statin, fibrate, antipsychotic, neuromuscular blocking agents, SSRIs, diuretic use, venom or heavy metal poisoning, infection and autoimmune disorders.

Examples of delivery routes include, without limitation: topical, for example, epicutaneous, inhalational, enema, ocular, otic and intranasal delivery; enteral, for example, orally, by gastric feeding tube or swallowing, and rectally; and parenteral, such as, intravenous, intraarterial, intramuscular, intracardiac, subcutaneous, intraosseous, intradermal, intrathecal, intraperitoneal, transdermal, iontophoretic, transmucosal, and epidural.

The compositions described herein are useful for treatment of fatty acid oxidation disorders. They have anti-inflammatory activities. The following are non-limiting descriptions of various criteria used to determine if a given composition produces a physiological effect in a patient.

### Inflammatory Response:

The inflammatory response (inflammation) occurs when tissues are injured by disease, congenital conditions, bacteria, trauma, toxins, heat, or any other cause. Systemic inflammation is the result of release of pro-inflammatory cytokines from immune-related cells and the chronic activation of the innate immune system. Pro-inflammatory cytokines are cytokines that are important in cell signaling and promote systemic inflammation. They are produced predominantly by activated macrophages and are involved in the upregulation of inflammatory reactions. In contrast to anti-inflammatory cytokines, which promote healing and reduce inflammation, pro-inflammatory cytokines can act to make a disease worse. For patients with harmful excess inflammatory reactions, anti-cytokine therapies include neutralizing antibodies, serving as soluble receptors and receptor antagonists, and inhibiting proteases. Pro-inflammatory cytokines arise from genes that induce a response after upregulation. Some examples of pro-inflammatory cytokines include: interleukins, such as, but not limited to, IL-1, IL-8, IL-17, interferons, such as, but not limited to, IFN-γ, IFN-α, and IFN-β, tumor necrosis factor, such as, but not limited to, TNF-α and TNF-β. Release of pro-inflammatory cytokines and activation of the innate immune system may be the result of either external (biological or chemical agents) or internal (genetic mutations/variations) factors, such as in the case of FAODs. Systemic inflammation is a hallmark of FAODs. As such, compositions that are capable of reducing the systemic inflammatory are required.

### Measuring Systemic Inflammation

Systemic inflammation, and the reduction thereof, may be measured by assessing levels of serum inflammatory markers (serum cytokines) and macrophage activation. Modulation of inflammatory cytokines can be assessed by determining the level of each cytokine in the plasma or by measuring the level of expression of the cytokines in the blood cells. Plasma cytokine levels are commonly determined, for example, by utilizing a cytokine detection kit, for example an ELISA (enzyme-linked immunosorbant assay) kit.

### Energy Metabolism

As described elsewhere herein, energy metabolism is the process of generating energy (ATP) from nutrients. Fatty acids are a family of molecules classified within the lipid macronutrient class. One role of fatty acids within animal metabolism is energy production, captured in the form of adenosine triphosphate (ATP). When compared to other macronutrient classes (carbohydrates and protein), fatty acids yield the most ATP on an energy-per-gram basis, when they are completely oxidized to CO₂ and water by β-oxidation and the citric acid cycle. Fatty acid oxidation disorders are caused by genetic disorders that ultimately result in a patient's inability to produce or utilize a required enzyme needed to oxidize fatty acids. As such, compositions that are capable of increasing and improving an FAOD patient's ability to oxidize fatty acids are required. Methods of measuring energy metabolism are described below, for example, feeding labeled fat (e.g. palmitate, palmitate oxidation) to determine how efficiently CO₂ is produced, and animals kept at 4° C. will quickly get cold and die if their energy production is insufficient.

### Antioxidant (Reactive Oxygen Species):

One measure of anti-inflammatory effect is the reduction of reactive oxygen species (ROS), which are chemically-reactive chemical species containing oxygen. Examples include peroxides, superoxide, hydroxyl radical, and singlet oxygen. The measurement of reactive oxygen species is dependent on the analytic target along with the reactive oxygen species in question. At the cellular level, specific ROS can be individually assessed from tissue culture, while at the animal level typically the effects of oxidative stress are measured from blood product (e.g. serum or plasma) or from urine samples. Various means for measuring ROS may be used, and are broadly-recognized in the art and are commercially available, e.g. from Enzo Life Sciences, Inc. (Farmingdale, N.Y.). Some non-limiting examples include: measurement of the ratio of reduced glutathione (GSH) is regenerated from its oxidized form (GSSH), e.g., by HPLC, capillary electrophoresis, or biochemically in microplates; superoxide detection, for example by fluorescent, chemiluminescent, or colorimetric detection, including hydrocyanine dyes (for superoxide and hydroxyl radical) such as hydro-Cy3 and hydro-Cy5, dihydroethidium, and MitoSOX™ Red reagent (Life Technologies); hydrogen peroxide (H₂O₂) detection, e.g., by use of fluorogenic substrates, e.g., diacetyldichloro-fluorescein, homovanillic acid, and Amplex® Red, which serve as hydrogen donors that have been used in conjunction with horseradish peroxidase (HRP) enzyme to produce intensely fluorescent products, or the oxidation of 2′-7′ dichlorofluorescin (H₂DCF) to 2′-7′dichlorofluorescein (DCF) has been used quite extensively for the quantitation of H₂O₂; and detection of the free radical nitric oxide (•NO) by determination of its products nitrate and nitrite colorimetrically or fluorescently.

### Example 1

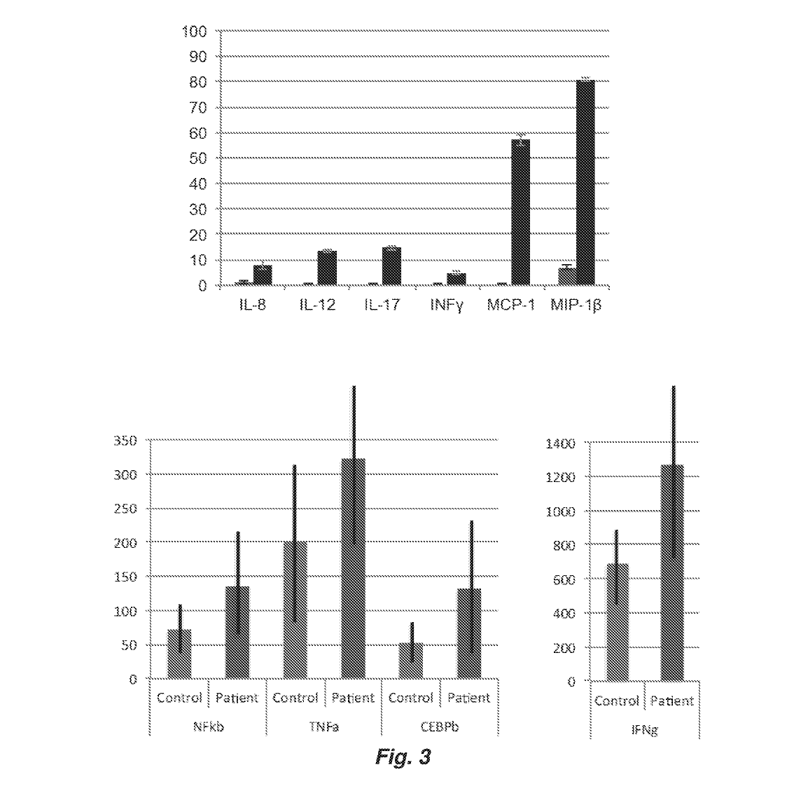

The aim of this Example is to test small molecule compounds, such as RTA408 and similar compounds, to treat long chain FAODs. Preliminary data demonstrated derangements both in energy metabolism and inflammation in patients with VLCAD deficiency. Treatment of patients having long chain FAODs with the anaplerotic agent triheptanoin essentially eliminates hypoglycemia in patients, but they continue to have episodes of rhabdomyolysis. Importantly, these patients show an inflammatory process consistent with macrophage activation irrespective of treatment with triheptanoin (FIG. 3).

VLCAD deficient mouse model and microarray expression studies have suggested an abnormal immune activation. To examine phenomenon more carefully, cytokine studies in euglycemic VLCADD mice without muscle symptoms, indicated up-regulated inflammatory pathways. VLCADD mice also showed a broad-based increase in pro-inflammatory cytokines in the. Cytokines were measured in blood of 8 VLCADD patients during a stable period, except for one patient who had a CPK>1,000. FIG. 3 provides preliminary evidence that VLCADD induces systemic inflammation as indicated by increased IL1β, IL6, IL17, IFNγ, TNFα and the inflammatory monokines MCP1 and MIPb.

The presence of high levels of IFNγ, MCP1, and GM-CSF suggests dysfunctional Th1-lymphocyte or monocyte response. Two patients have considerable elevations of all inflammatory cytokines including TNFα, L6, IL12, IL17, and IFNγ. TNFα is myolytic, while IL6 attenuates muscle contraction. Co-elevation of TNFα and IL6 has been correlated with frailty syndrome and sarcopenia. One patient has elevated Th2 cytokines (IL4, IL5, IL6, L13), indicating either an allergic symptomatology or hyperglobulinemia. These results suggest long-chain FAODs pathophysiology may involve inflammation.

### Example 2

Recently the stress-associated energy problem in VLCAD defective subjects has been addressed by supplementing them with intravenous glucose to supply energy, along with chronic triheptanoate to generate anaplerotic tricarboxylic acid cycle precursors to improve efficiency. In spite of improved energy management, these patients still present intermittently with life-threatening episodes of decompensation or rhabdomyolysis. The underlying cause of these remaining problems is not readily apparent, but we reasoned that these morbidities might be associated with fatty acid alterations affecting other non-energetic metabolic functions of fats, such as signaling or membrane structure.

One means to investigate the origins of these complex residual morbidities of unknown etiology is to use non-hypothesis driven methods that interrogate a broad swath of potential alterations with one methodology. We choose to try gene expression array and blood cytokine analysis to give us more insight into the effects of VLCAD deficiency in a mouse model.

### Materials and Methods:

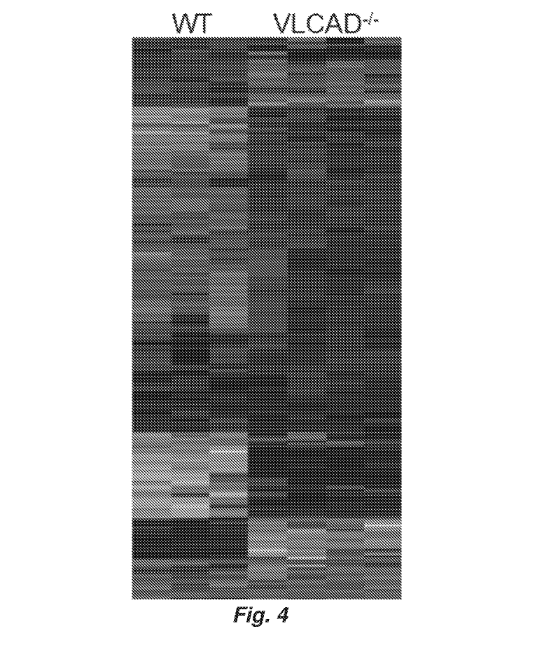

Microarray expression analysis was conducted (Affymetrix Genome 430A 2.0 with 14,000 genes/22,690 probes), comparing well-controlled VLCADD (knockout mice with corresponding wild-type background animals in 4 tissue types (cardiac, liver, brown fat, and skeletal muscle), and was used initially to generate a heat map of differences between the two strains, and identify candidate genes to define pathogenesis and target therapy. As shown in FIG. 4, a pattern suggesting immune activation was identified, so inflammatory cytokine profiles were examined in VLCADD mice and patients were analyzed by Bio-Plex Pro Mouse Cytokine 23-plex and Human 17-plex (BioRad) Luminex. Patients, evaluated included 9 females and 8 males, ages 1 week to 34 years old. 14 serial samples were obtained during three rhabdomyolysis episodes. Monocyte phenotypes in patients were examined by multicolor flow cytometry.

### Results and Discussion:

Mouse models of long chain fatty acid oxidation. RNA expression arrays were performed from various tissues from unstressed. VLCAD deficient mice. 30 genes associated with immune function showed at least 2-fold elevation in the VLCADD mice as compared to wild-type. These genes can be further subdivided into interferon-mediated (2 genes), local T-cell mediated (8 genes), macrophage-mediated inflammation (10 genes) and innate-mediated inflammation via complement and alternate pathways (6 genes) and local tissue destruction (4 genes).

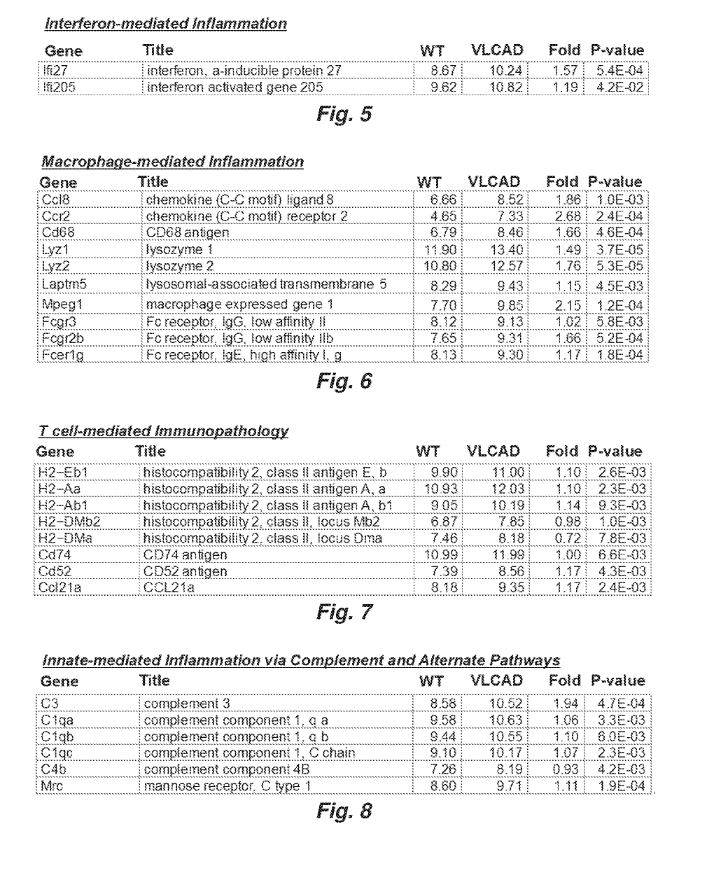

FIGS. 5-8 provide a comparison of VLCAD deficient animals to wild type animals should that the genes involved in the inflammatory response are elevated.

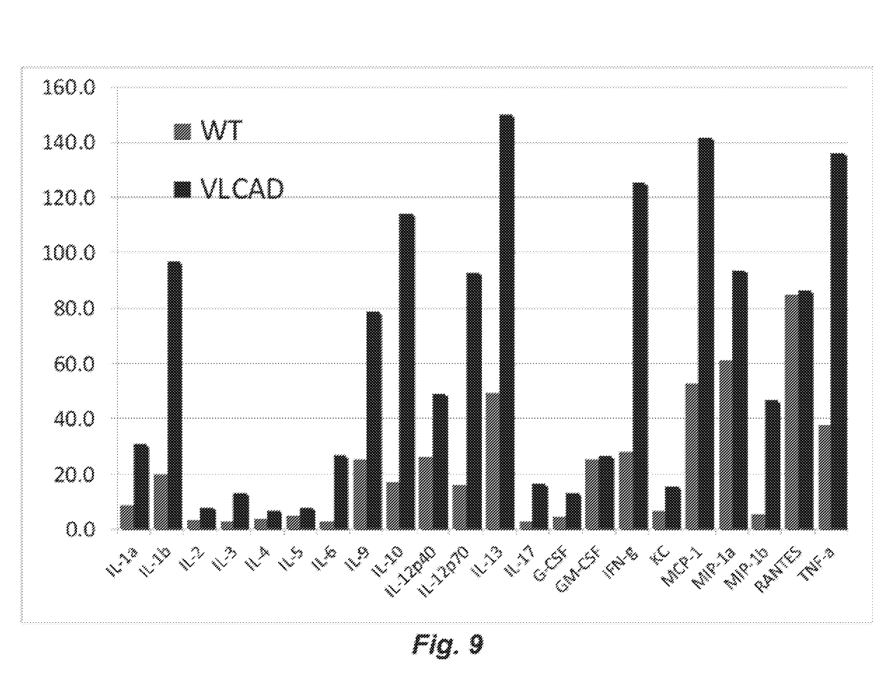

Cytokine analysis of serum from wild type and VLCAD deficient mice shows that multiple cytokines are elevated in VLCADD mice not on dietary therapy, including IL-1β, IL-3, IL-6, IL-10, IL-12, IL-17, INFγ, and MIP-1β (FIG. 9). Similarly, elevations of IL-8, IL-12, IL-17, INFγ, MCP-1, and MIP-1β were observed in VLCADD patients regardless of dietary therapy or hospitalization.

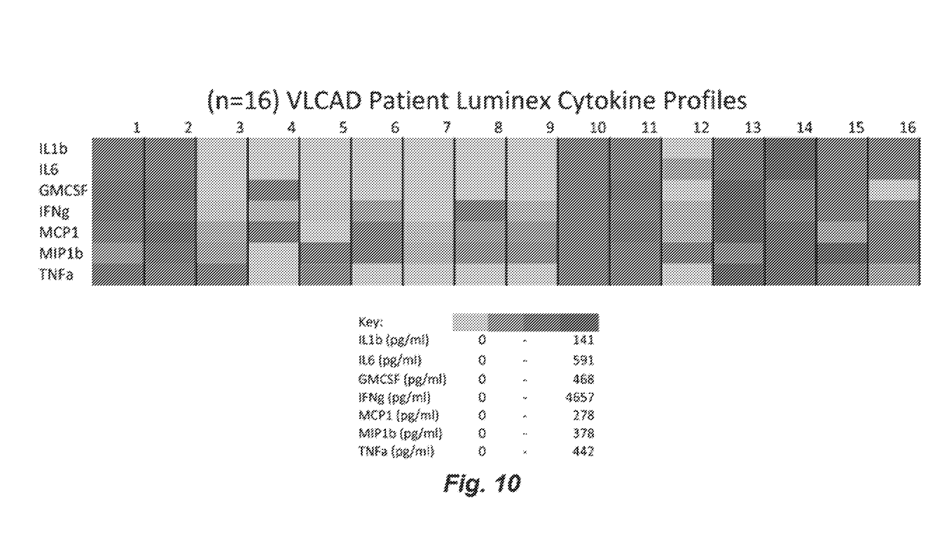

Luminex profile of cytokines from (n=16) VLCAD patients show that eight of them exhibit increased concentrations of cytokines (≥100 pg/ml; FIG. 10).

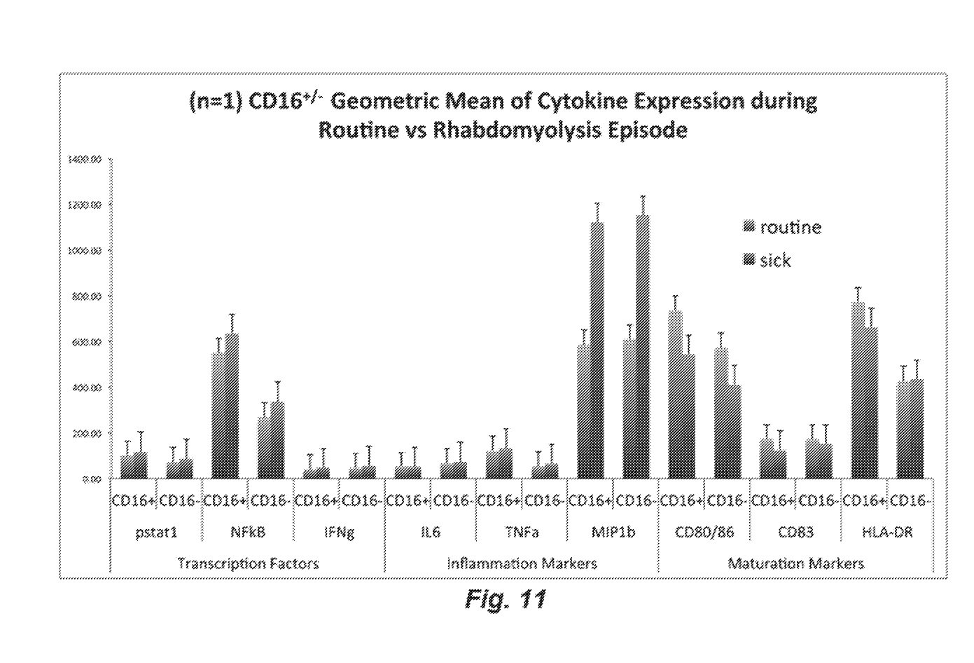

Mean fluorescence intensities of cytokines in CD16+/− cells of a VLCAD patent with recurring rhabdomyolysis shows increased intensities in blood monocytes (FIG. 11).

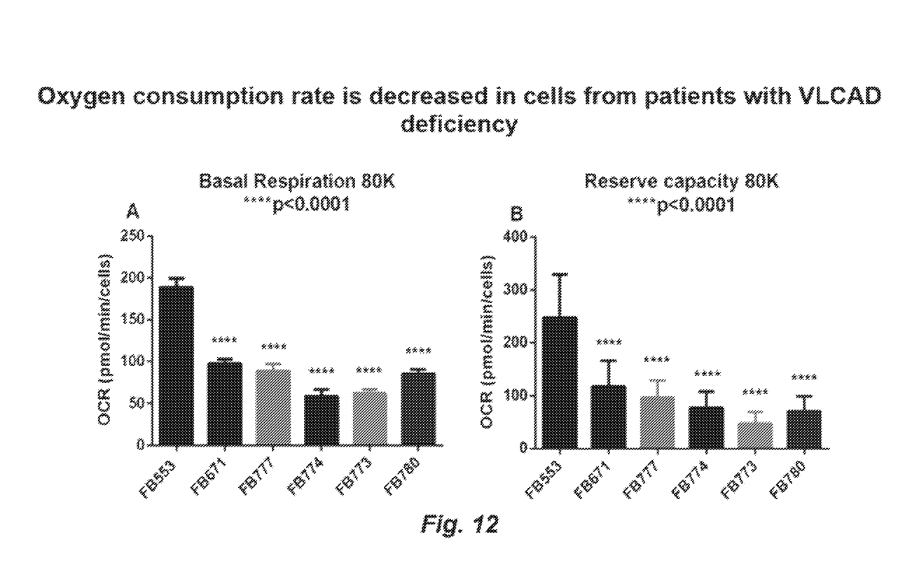

Measurement of whole cell oxygen consumption in VLCAD and LCHAD deficient cell lines identifies that an increased concentration of reactive oxygen species in cells as measured by MitoSox and flow cytometry leads to reduced basal cellular respiration and reserve capacity as demonstrated by whole cell oxygen consumption as measured by a Seahorse apparatus (FIG. 12).

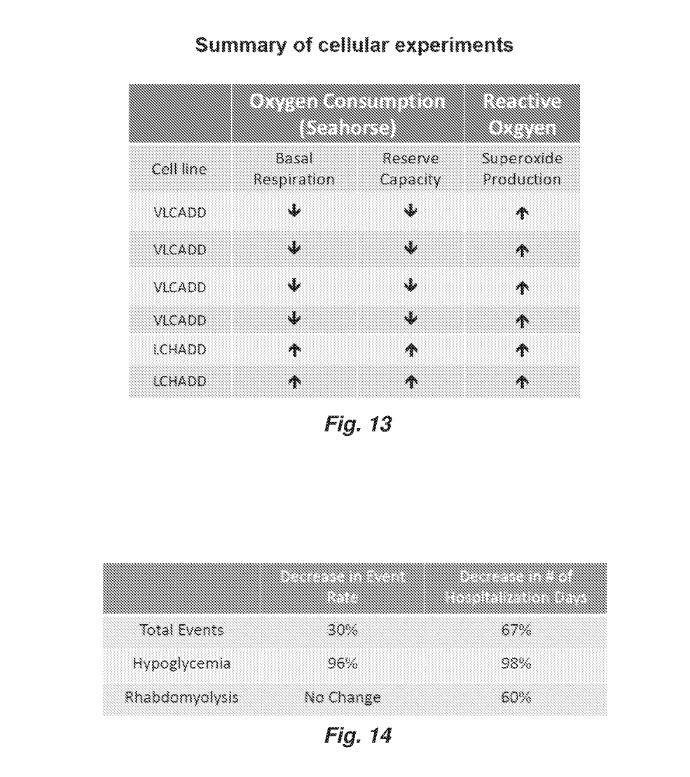

FIG. 13 is a summary of the cellular experiments illustrating that overall oxygen consumption is reduced in VLCAD deficient cell lines, whereas oxygen consumption is increased in LCHAD deficient cell lines. Reactive oxygen species are increased in both disorders.

In total, these data indicate that disorders in long chain fatty acid oxidation induce to an atypical inflammatory response that affects energy function over and above that caused by the primary deficiency, likely leading to or exacerbating the rhabdomyolysis seen in these patients. Reduction of this inflammatory process will provide significant benefit to patients by reducing frequency and severity of episodes of rhabdomyolysis.

### Example 3

Long-chain fatty acid oxidation disorders (LC-FAOD's) can lead to frequent complications and the need for hospitalizations of patients. Treatment of patients suffering from LC-FAOD's with triheptanoin reduced hospitalizations and if hospitalized, the number of days of hospital stay. Hypoglycemic hospitalizations were nearly eliminated. With rhabdomyolysis, the number of hospitalizations did not reduce, but the number of hospitalization days decreased. See FIG. 14.

### Example 4

It is hypothesized that the residual inflammation, and/or secondary changes induced in patients with FAODs, are responsible for the continued rhabdomyolysis. It also is hypothesize that additional therapeutic molecules have the potential to stabilize or induce mutant fatty acid oxidation proteins and mitigate the atypical inflammatory process seen in FAODs deficiencies, offering novel therapeutic options for patients with these diseases.

A first aim of this example is to examine the effect of candidate therapeutic compounds on fatty acid oxidation function in cells from patients with FAODs.

Experimental Approach.

Repurposing of available medications or compounds in clinical trials for treatment of FAODs has the benefit of requiring less safety and preclinical testing before such drugs can be tested in patients. This example evaluates the effect on fatty acid oxidation of experimental therapeutic compounds for treatment of mitochondrial respiratory chain deficiency. These compounds induce expression of the proteins involved in mitochondrial energy metabolism (including FAO enzymes) and down-regulate key inflammatory pathways that are aberrantly stimulated in these disorders. It is believed that induction of FAO enzymes and reduction of inflammation are likely to be beneficial to patients with long chain FAODs as our preliminary findings are consistent with an inflammatory phenotype in VLCAD patients. Human clinical trials in patients with long chain FAODs are premature given the lack of preclinical data on their effects in these disorders, so cellular and animal studies are proposed to obtain the necessary preclinical data to conduct such trials.

Various assays, as follow, are routine in my lab that allow assessment of overall energy metabolism in cells and more specifically interrogate FAO function. Cells from patients with FAODs are treated with candidate compounds (up to six) at a range of concentrations selected empirically or on the basis of company preliminary data. Initial experiments focus on cells with CPT2, VLCAD, and LCAHD mutations, but can be expanded to the other long chain FAODs. Prior to treatment, cells are tested for baseline gene expression and residual stability of each mutant enzyme. After treatment with the experimental agent, cells are tested for change in FAO/enzyme function, membrane stability, and cell viability as described below (Chen M, et al. (2011). Mitochondria-targeted peptide MTP-131 alleviates mitochondrial dysfunction and oxidative damage in human trabecular meshwork cells. Invest Ophthalmol Vis Sci. 52: 7027-37). Additionally, cells are tested for induction of apoptosis using in situ assay for caspase-3 induction (BioRad FITC-VAD-FMK and Anti-ACTIVE Caspase-3 antibody). A Seahorse apparatus is used to measure whole cell oxygen consumption and glycolysis using standard lab procedures. Assays are repeated following cell stress induced by hydrogen peroxide, low glucose/high galactose, and high palmitate exposure. Treated cells are additionally tested for expression of FAO and inflammatory protein gene expression using standard protocols (Goetzman E S, et al. (2007). Expression and characterization of mutations in human very long-chain acyl-CoA dehydrogenase using a prokaryotic system. Molecular Genetics and Metabolism. 91: 138-47; Maher A C, et al. (2010). Low expression of long-chain acyl-CoA dehydrogenase in human skeletal muscle. Molecular Genetics and Metabolism. 100: 163-7; He M, et al. (2011). Identification and characterization of new long chain Acyl-CoA dehydrogenases. Mol Genet Metab. 102: 418-29; Graves J A, et al. (2012). Mitochondrial Structure, Function and Dynamics Are Temporally Controlled by c-Myc. PloS one. 7: e37699; Schiff M, et al. (2013). Molecular and cellular pathology of very-long-chain acyl-CoA dehydrogenase deficiency. Mol Genet Metab. 109: 21-7; Schiff M, et al. (2015). Complex I assembly function and fatty acid oxidation enzyme activity of ACAD9 both contribute to disease severity in ACAD9 deficiency. Hum Mol Genet. 24: 3238-47).

### Specific Techniques

Preparation of Mitochondria from Rat Liver.

Freshly isolated rat liver is immediately homogenized in a tissue blender at high speed for 20 sec at 4° C. in a buffer containing 25 mM Tris-HCl, pH 7.5; 100 mM KCl; 0.4 M sucrose; and protease inhibitor cocktail (Sigma, St. Louis, Mo.). The homogenate was centrifuged at 900×g for 10 min at 4° C., and the pellet was discarded. The supernatant containing mitochondria was subjected to centrifugation at 14,000×g for 15 min, the pellet was washed once with the homogenization buffer, and the mitochondria collected by centrifugation under the same conditions, and resuspended in the same buffer.

Blue Native Gel Electrohoresis (BNGE.

Isolated mitochondria (100 μl, 1 mg of protein) are lysed by the addition of 200 μl of high purity digitonin solution (1:4 gm protein:gm digitonin) prepared as follows: 5 mg of digitonin (MP Biomedicals, Solon, Ohio) are dissolved in 200 μl of 30 mM HEPES buffer, pH 7.4 containing 150 mM potassium acetate and 10% glycerol, heated at 95° C., then cooled on ice. The mitochondria/digitonin solution (final protein concentration 3 mg/ml) is incubated for 20 min on ice, a Coomassie blue solution (5% coomassie blue G250 in 750 mM 6-aminocaproic acid) was added (1/20 v/v), and the reaction mixture is centrifuged at 14,000×g for 30 min at 4° C. The supernatant is directly loaded onto a 4-15% Tris-HCl Ready Gel (Bio-Rad, Hercules, Calif.), pH 8.8 and subjected to electrophoresis in 25 mM Tris-glycine buffer (without SDS) at 80 constant voltage for 4 h, at 4° C. Following electrophoresis, gels are stained with Bio-Safe Coomassie G250 (Bio-Rad, Hercules, Calif.) for 30 min and destained with water. For second dimension separation, a strip of the gel corresponding to a single sample well is rotated 90 degrees and placed on a 12% SDS-PAGE gel and subjected to electrophoresis as described (Reifschneider N H, Goto S, Nakamoto H, Takahashi R, Sugawa M, Dencher N A, Krause F. (2006). Defining the mitochondrial proteomes from five rat organs in a physiologically significant context using 2D blue-native/SDS-PAGE. J Proteome Res. 5: 1117-32).

Western Blotting.

Following BNGE or SDS-PAGE, proteins in the gel are electrophoretically transferred to PVDF membrane for 2 hr as described (Schagger H, et al. (1994). Analysis of molecular masses and oligomeric states of protein complexes by blue native electrophoresis and isolation of membrane protein complexes by two-dimensional native electrophoresis. Analytical Biochemistry. 217: 220-30). The membrane is blocked for 1 hr at room temperature in 20 ml phosphate buffered saline containing 0.1% Tween 20 and 7% powdered milk. The primary antiserum is diluted 1:500-1000 in the same buffer while the secondary antisera are diluted 1:2000-5000 (goat anti-rabbit IgG conjugated to alkaline phosphatase) and 1:5000 (goat anti-rabbit IgG conjugated to horseradish peroxidase).

In Situ Gel Staining for ETC-Enzyme Activity.

Strips corresponding to the sample wells re excised from the blue native gels and incubated with reaction buffer systems specific for ETC complexes 1, II, IV or V. The Activity staining procedures are essentially as described in Van Coster R, et al. (Van Coster R, et al. (2001). Blue native polyacrylamide gel electrophoresis: a powerful tool in diagnosis of oxidative phosphorylation defects. Pediatr Res. 50: 658-65).

Enzyme Activity Assay.

ACAD activity is measured with the anaerobic ETF fluorescence reduction assay using an LS50B fluorescence spectrophotometer from Perkin Elmer (Norwalk, Conn.) with a heated cuvette block set to 32° C. as previously described (Van Coster R, et al. (2001). Blue native polyacrylamide gel electrophoresis: a powerful tool in diagnosis of oxidative phosphorylation defects. Pediatr Res. 50: 658-65; Vockley J, et al. (2000). Mammalian branched-chain acyl-CoA dehydrogenases: molecular cloning and characterization of recombinant enzymes. Methods Enzymol. 324: 241-58; Mohsen A W, et al. (1998). Characterization of molecular defects in isovaleryl-CoA dehydrogenase in patients with isovaleric acidemia. Biochemistry. 37: 10325-35). For standard reactions, the final substrate concentration is 50 μM. One unit of activity is defined as the amount of enzyme necessary to completely reduce 1 μmol of ETF in 1 minute. Additional enzyme assays are as described (Wanders R J, et al. (2010). The enzymology of mitochondrial fatty acid beta-oxidation and its application to follow-up analysis of positive neonatal screening results. Journal of Inherited Metabolic Disease. 33: 479-94).

ETC complex I activity is measured in 50 mM phosphate buffer, pH 7.4, 50 μM NADH, 1 mM KCN, and 50 μM oxidized Coenzyme Q as described (Wang Y, et al. (1995). Topographical organization of cytochrome b in the yeast mitochondrial membrane determined by fluorescence studies with N-cyclohexyl-N′-[4-(dimethylamino)naphthyl]carbodiimide. Biochemistry. 34: 7476-82; Fang J, et al. (2001). Isolation and characterization of complex I, rotenone-sensitive NADH: ubiquinone oxidoreductase, from the procyclic forms of Trypanosoma brucei. Eur J Biochem. 268: 3075-82). The reaction is detected by monitoring the decrease in absorbance at 340 nm and quantified using a molar extinction coefficient of 6.2 mM⁻¹cm⁻¹. Complex III activity (defined as cytochrome c reduction) is measured in 50 mM phosphate buffer, pH 7.4, 1 mM KCN, 30 μM cytochrome c, 20 μM DBH₂, monitoring the increase in absorbance at 550 nm (Kniazeva M, et al. (2004). Monomethyl branched-chain fatty acids play an essential role in Caenorhabditis elegans development. PLoS Biol. 2: E257; Fraser J L, et al. (1995). Prevalence and Nonspecificity Of Microvesicular Fatty Change In the Liver. Modern Pathology. 8: 65-70). Complex IV activity is measured in 50 mM phosphate buffer, pH 7.4, 6 μM ferrocytochrome c, 100 mM KCl. The reaction is monitored at 550 nm (Prochaska L J, et al. (1981). Inhibition of cytochrome c oxidase function by dicyclohexylcarbodiimide. Biochim Biophys Acta. 637: 360-73).

FAQ-ETC Bridging Assay.

This reaction reflects the interaction of FAO and ETC as measured by reduction of cytochrome c in response to the addition of an acyl-CoA substrate to the reaction mixture. The basic reaction scheme is as follows: an aliquot of sample (usually the sucrose gradient supercomplex fraction) is added to reaction buffer (50 mM phosphate, pH 8.2 containing 50 μM acyl-CoA substrate, 3 μM ETF, 20 μM oxidized coenzyme Q, 30 μM cytochrome c, 1 mM KCN) to give a final volume of 0.7 ml. The reaction is started by the addition of ETF and monitored for the reduction of cytochrome c as indicated by an increase in absorbance at 550 nm using a Beckman DU7500 spectrophotometer (Wang Y, et al. (1998). Dicyclohexylcarbodiimide inhibits proton pumping in ubiquinol:cytochrome c oxidoreductase of Rhodobacter sphaeroides and binds to aspartate-187 of cytochrome b. Arch Biochem Biophys. 352: 193-8). Stearyl-CoA, palmitoyl-CoA, octanoyl-CoA, or butyryl-CoA is utilized as substrate to test the long, medium, and short chain specificity of the assay, respectively. A variety of enzymatic inhibitors are added to the base reaction to characterize the contribution of reducing equivalents from reduced ETF and NADH to cytochrome c reduction. Myxothiazol and antimycin A inhibit complex III. Rotenone is a specific inhibitor of complex I. 3-Mercaptopropionic acid (MPA) inhibits long chain ACADs (Uchiyama A, et al. (1996). Molecular Cloning of cDNA Encoding Rat Very Long-Chain Acyl-Coa Synthetase. J Biol Chem. 271: 30360-5). Kinetic parameters for cytochrome c reduction are calculated using a non-linear regression algorithm as previously described (Wang Y, et al. (1998). Dicyclohexylcarbodiimide inhibits proton pumping in ubiquinol:cytochrome c oxidoreductase of Rhodobacter sphaeroides and binds to aspartate-187 of cytochrome b. Arch Biochem Biophys. 352: 193-8; Trumpower B L. (1990). The protonmotive Q cycle. Energy transduction by coupling of proton translocation to electron transfer by the cytochrome bc1 complex. J Biol Chem. 265: 11409-12).

High Pressure Liquid Chromatographv (HPLC) FAO Flux Assay.

The ability of a sample to perform all of the functions of the entire FAO pathway is tested by following depletion of an acyl-CoA substrate and accumulation of various cycle intermediates via HPLC. Sucrose gradient fractions (2 μg of protein) or purified recombinant ACAD (2-5 ng protein) re incubated with 50-300 μM butyryl-, octanoyl- or palmitoyl-CoA at room temperature for 0-50 min. The reaction contains 1 mM μg ETF, 1.5 mM NAD, 0.3 mM CoASH and 0.2 mM ATP. The reaction is stopped at 5 min intervals by the addition of 40 ml of 2 N HCl, and then immediately neutralized with 40 ml of 2 N NaOH in 200 mM MES. Following clearing of the sample in a bench top microcentrifuge for 10 min, 150 μl of sample are injected into a Luna (Phenomenex, Torrance, Calif.) 3 μm Cl 8(2), 100 Å, 4.6×150 mm, 3 μm column (for palmitoyl-CoA reactions) or a SunFire (Phenomenex Torrance, Calif.) C18, 3.5 μm column (for octanoyl- and butyryl-CoA reactions) equilibrated with 50 mM NH₄PO₄ pH 5.5 with 9% acetonitrile. The CoA esters that bound to the columns are eluted with a 9-45% linear gradient of acetonitrile in 50 mM NH₄PO₄ pH 5.5 at 0.9 ml/min over 50 min (Luna column) and 25 minutes (SunFire column). The presence of CoA ester intermediates in the eluent is monitored by absorbance at 254 nm and compared to the retention times of acyl-CoA ester standards eluted under identical conditions.

Whole Cell Oxidation and Glycolysis Studies (Seahorse).

Oxygen consumption rate (OCR) and extracellular acidification rate (ECAR) measurements will be performed with a Seahorse XFe96 Extracellular Flux Analyzer (Seahorse Bioscience, MA). By measuring OCR and ECAR, the analyzer assesses in vitro oxidative phosphorylation (OXPHOS) and glycolysis, respectively, in real time. Cells are seeded in 96-well Seahorse tissue culture microplates previously coated with Corning® Cell-Tak™ Cell and Tissue Adhesive in order to attach them at a density of 8×10⁴ cells per well. After 15-25 min, the plates are incubated in unbuffered DMEM without CO₂ for 1 h at 37° C. and then taken to the Seahorse Analyzer. First, the Seahorse Analyzer will measure OCR and ECAR baselines, and afterwards, additional automated measurements will be performed after the injection of four compounds affecting bioenergetic capacity: oligomycin (1 μM; inhibitor of respiratory chain complex V activity), FCCP (300 nM; classical uncoupler of respiratory chain), 2-deoxyglucose (100 mM; inhibitor of glycolysis), and rotenone (1 μM; inhibitor of respiratory chain complex I). Data will be normalized to cell number and expressed as pmol/min/cell for OCR and mpH/min/cell for ECAR.

Evaluation of Cell Viability.

Cell viability is evaluated by measuring LDH released from the cells with a calorimetric assay (Cytotoxicity Detection KitPLUS; Roche Diagnostics) according to the manufacturer's instructions. Cells are plated in 96-well plates at a density of 1×10⁴/well for 24 hours. After being pretreated with MTP-131 for 1 hour, cells are incubated with 200 μM H₂O₂ for 24 hours. Culture supernatant of each well (50 μL) is transferred to a fresh 96-well plate and 100 μL reaction mixture solution is added to each well and incubated at room temperature for another 30 minutes in the dark. Termination is achieved by adding 50 μL stop solution to each well. The microplate is read at absorbance at 490 nm (Benchmark Microplate Reader, Bio-Rad, Hercules, Calif.). Cell viability is calculated as follows: cytotoxicity (%)=[(experimental value−low control)/(high control−low control)]×100%.

Measurement of Change in Membrane Potential (ΔΨm)

ΔΨm was evaluated with fluorescent probe TMRM. Cell cultures (1×10⁵ cells/well in a 6-well plate) are pretreated with MTP-131 for 1 hour and then incubated with 200 μMH₂O₂ for 2, 4, and 8 hours. At subsequent time points, cells are harvested and suspended in freshly prepared TMRM (100 nM) in phenol and serum-free DMEM/F12 for 30 minutes at 37° C. in the dark. Samples are rinsed twice with PBS and analyzed immediately by flow cytometry (excitation/emission 548/573 nm). Ten thousand cells are routinely collected, and the mean fluorescence intensity (MFI) in arbitrary units (AU) from at least three separate experiments was analyzed. For confocal microscopy, cells are plated in Petri dishes. After various treatments, cells were rinsed twice and loaded with 500 nm TMRM for 30 minutes at 37° C. in the dark followed by a 5-minute incubation with Hoechst 33342 (1:1000; Molecular Probes/Invitrogen). After being rinsed twice with PBS, cells are imaged by confocal microscopy using Z-stack mode (LSM 510; Carl Zeiss, Germany; excitation/emission 548/573 nm).

Measurement of Intracellular ROS.

Intracellular ROS is evaluated with the fluorescent probe H₂DCFDA. Briefly, cells re pretreated with MTP-131 for 1 hour and incubated with 200 μMH₂O₂ at 37° C. for 24 hours. Cells are harvested and suspended in freshly prepared 1 μM H₂DCFDA at 37° C. for 30 minutes in the dark. After being rinsed twice with PBS, cells are analyzed immediately by flow cytometry (excitation/emission 488/530 nm). Ten thousand cells are routinely collected, and the MFI in AU from at least three independent experiments was analyzed. For visualization by confocal microscopy, cells are plated in Petri dishes. After pretreatment with MTP-131 and incubation with H₂O₂, cells are washed and loaded with 5 μM H₂DCFDA at 37° C. for 30 minutes in the dark. After being rinsed twice with PBS, cells are imaged by confocal microscopy (excitation/emission 495/525 nm).

Detection of Apoptosis by Annexin V and PI.

Apoptosis is quantified by flow cytometry. Briefly, cells are pretreated with MTP-131 for 1 hour and then incubated with 200 μM H₂O₂. At the subsequent 4-, 8-, 12-, and 24-hour time points, cells are collected and incubated with 5 μL Annexin V and 10 μL PI according to the manufacturer's instructions. Annexin V-FITC is analyzed by flow cytometry (excitation/emission 488/530 nm) using the FITC channel (FL1) and PI staining was analyzed by the PE channel (FL2). Cells are distinguished as viable (Annexin V−/PI−, Q3), early apoptotic (Annexin V+/PI−, Q4), late apoptotic (Annexin V+/PI−, Q2), or dead (Annexin V−/PI+, QI) cells. The apoptotic rate was calculated as the percentage of early apoptotic cells (Q4%) plus the percentage of late apoptotic cells (Q2%).

Measurement of Cytochrome c Release.

Release of cytochrome c from mitochondria to cytoplasm is measured by confocal microscopy. Cells are seeded onto growth cover glasses at a density of 2000 cells/chamber. After being pretreated with MTP-131 for 1 hour, cells were incubated with 200 μM H₂O₂ for 6 hours. Freshly prepared intensive red fluorescence dye (200 nm) is loaded into cells in advance and incubated at 37° C. for another 30 minutes. Then the cells re fixed in 4% paraformaldehyde (PFA) for 15 minutes and permeabilized in methanol for 5 minutes on ice. After being blocked with 5% BSA for 30 minutes, cells are incubated with mouse monoclonal anti-cytochrome c (1:100; Santa Cruz Biotechnology, Santa Cruz, Calif.) at 4° C. overnight. After being rinsed with PBS three times, cells are incubated with conjugated goat anti-mouse IgG (Dylight 488, 1:500; Multisciences) for 30 minutes at 37° C. in the dark, followed by Hoechst 33342 for 5 minutes, then washed and mounted with antifade fluorescence mounting medium (Applygen Technologies, Beijing, China). Translocation of cytochrome c from mitochondria to cytoplasm is analyzed by overlapping of co-stained cytochrome c and highly intense red fluorescent dye.

Measurement of Caspase-3 by Western Blotting.

Cells at a density of 2×10⁵ cells/mL are plated onto 100-mm plates and incubated at 37° C. for 48 hours. After being pretreated with MTP-131 for 1 hour, cells are incubated with 200 μM H₂O₂ for another 12 hours. Then, cells are rinsed and lysed in mammalian cell lysis reagent (Sigma-Aldrich) containing protease inhibitor cocktail (Sigma-Aldrich). Samples (20 μg) are loaded onto a 10% SDS-PAGE gel and electrophoresed at 150 V for 1 hour. The membrane is then visualized with anti-caspase 3 monoclonal antibody (1:1000; Cell Signaling Technology, Danvers, Mass.) and anti-β-actin antibody (1:1000; Multiscience Biotech, Hangzhou, China). The protein levels are normalized by α-actin

Anticipated Results and Interpretation.

An improvement in one or more of the processes examined in cellular studies are to be taken as a positive result to consider for future clinical trials. It will not be necessary to perform all tests with all compounds on all cell lines. The first cell line can be analyzed extensively and the best suite of procedures to demonstrate a change with the test compounds can be used thereafter. It is predicted that FAO genes will be upregulated by the test compounds and that some mutations will lead to increased stable and active protein in cells. Inflammatory genes are not active in fibroblasts and thus no effect of RTA408 on expression of these genes is likely.

A second aspect of this example is to examine the effect of candidate therapeutic compounds in an in vivo mouse models of FAODs in order to prepare for possible clinical trials in patients.

Experimental Approach:

Fibroblasts cannot be tested for anti-inflammatory effects, nor is it certain that membrane or enzyme stability effects will translate into a similar response in muscle tissue. Therefore, the compounds can also be tested in FAOD mouse models. Animals and controls will be given drug via food or oral gavage as appropriate at concentrations suggested by cell studies. They are then be tested for evidence of rhabdomyolysis (plasma CK and myoglobin), serum inflammatory markers (serum cytokines), and macrophage activation as in our preliminary experiments. Animals are placed under physiologic stress in a 4° C. cold room and monitored for ability to maintain body temperature, a standard test for FAO function in mice. Finally, animals are sacrificed and skeletal and cardiac muscle will be examined for basic histology looking for evidence of muscle damage or cardiomyopathy. Enzyme activity, integrity of the multifunctional energy complex, and cellular evidence of apoptosis is measured as above.

Anticipated Results and Interpretation.

It is predicted that candidate compounds will exhibit positive effects on cellular and mitochondrial stability and reduce physiologic stress-induced changes with prevention of rhabdomyolysis due to a decrease the inflammatory response. All of our mouse models are knock outs so the transcriptional activation of FAO genes is not likely to be relevant, though a stimulation of the respiratory chain may have secondary beneficial bioenergetic effects.

Various aspects of the invention are described in the following clauses:

Clause 1. A method of treating a long-chain fatty acid oxidation disorder, comprising administering to a patient a mitochondrial antiinflammatory composition, that optionally up-regulates fatty acid metabolism in a patient or stabilizes mitochondrial structure, in an amount effective to improve fatty acid oxidation function, improve mitochondrial respiratory chain function, increase mitochondrial stability, treat rhabdomyolysis, and/or decrease inflammation in a patient.

Clause 2. A method of treating inflammation associated with a long-chain fatty acid oxidase disorder, comprising administering to a patient a mitochondrial antiinflammatory composition, that optionally up-regulates fatty acid metabolism in a patient or stabilizes mitochondrial structure, in an amount effective to improve fatty acid oxidation function, improve mitochondrial respiratory chain function, increase mitochondrial stability, treat rhabdomyolysis, and/or decrease inflammation in a patient.

Clause 3. A method of treating inflammation associated with rhabdomyolysis associated with a long-chain fatty acid oxidation disorder, comprising administering to a patient a mitochondrial antiinflammatory composition, that optionally up-regulates fatty acid metabolism in a patient or stabilizes mitochondrial structure, in an amount effective to improve fatty acid oxidation function, improve mitochondrial respiratory chain function, increase mitochondrial stability, treat rhabdomyolysis, and/or decrease inflammation in a patient.

Clause 4. The method of clause 3, wherein the fatty acid oxidation disorder is rhabdomyolysis, and the treatment improves a symptom of rhabdomyolysis.

Clause 5. The method of any one of clauses 1-4, wherein the composition comprises D-Arg-2′6′-Dimethyltyrosine-Lys-Phe-NH₂, or an analog thereof, or a pharmaceutically-acceptable salt or ester thereof.

Clause 6. The method of any one of clauses 1-4, wherein the composition comprises D-Arg-2′6′-Dimethyltyrosine-Lys-Phe-NH₂, or a pharmaceutically-acceptable salt or ester thereof.

Clause 7. The method of any one of clauses 1-4, wherein the composition comprises

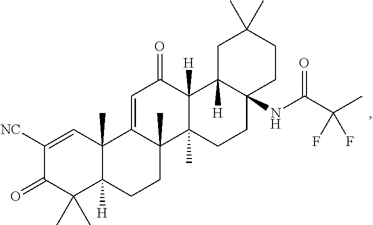

or an analog thereof, or a pharmaceutically-acceptable salt or ester thereof.

Clause 8. The method of any one of clauses 1-4, wherein the composition comprises:

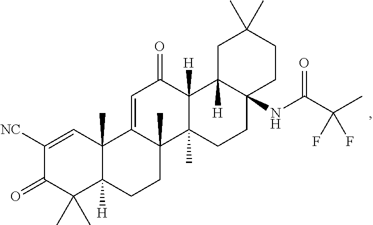

or a pharmaceutically-acceptable salt or ester thereof.

Clause 9. The method of any one of clauses 1-8, wherein the composition comprises one or more of triheptanoin or uridine triacetate.

Clause 10. A method of treating a fatty acid oxidation disorder in a patient, comprising administering to a patient an amount of a composition comprising D-Arg-2′6′-Dimethyltyrosine-Lys-Phe-NH₂, or a pharmaceutically-acceptable salt or ester thereof, effective to improve fatty acid metabolism and/or decrease inflammation in a patient.

Clause 11. A method of treating a fatty acid oxidation disorder in a patient, comprising administering to a patient an amount of a composition comprising

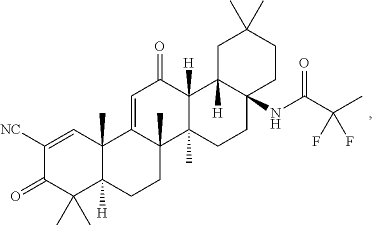

or a pharmaceutically-acceptable salt or ester thereof, effective to improve fatty acid metabolism and/or decrease inflammation in a patient.

Clause 12. A mitochondrial antiinflammatory composition that optionally up-regulates fatty acid metabolism in a patient or stabilizes mitochondrial structure, in an amount effective to improve fatty acid oxidation function, improve mitochondrial respiratory chain function, increase mitochondrial stability, treat rhabdomyolysis, and/or decrease inflammation in a patient for use in treating a long-chain fatty acid oxidation disorder, treating inflammation associated with a long-chain fatty acid oxidase disorder, and/or treating inflammation associated with rhabdomyolysis associated with a long-chain fatty acid oxidation disorder.

Clause 13. The mitochondrial antiinflammatory composition of clause 12, wherein the fatty acid oxidation disorder is rhabdomyolysis, and a symptom of rhabdomyolysis is improved.

Clause 14. The mitochondrial antiinflammatory composition of clause 12 or 13, comprising D-Arg-2′6′-Dimethyltyrosine-Lys-Phe-NH₂, or an analog thereof, or a pharmaceutically-acceptable salt or ester thereof.

Clause 15. The mitochondrial antiinflammatory composition of clause 12 or 13, comprising

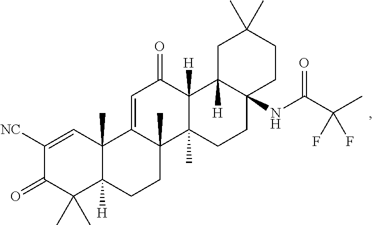

or an analog thereof, or a pharmaceutically-acceptable salt or ester thereof.

While several examples and embodiments of the methods are described hereinabove in detail, other examples and embodiments will be apparent to, and readily made by, those skilled in the art without departing from the scope and spirit of the invention. For example, it is to be understood that this disclosure contemplates that, to the extent possible, one or more features of any embodiment can be combined with one or more features of any other embodiment. Accordingly, the foregoing description is intended to be illustrative rather than restrictive.

1. A method of treating a long-chain fatty acid oxidation disorder, comprising administering to a patient a mitochondrial antiinflammatory composition, that optionally up-regulates fatty acid metabolism in a patient or stabilizes mitochondrial structure, in an amount effective to improve fatty acid oxidation function, improve mitochondrial respiratory chain function, increase mitochondrial stability, treat rhabdomyolysis, and/or decrease inflammation in a patient.

2. The method of claim 1, for treating inflammation associated with a long-chain fatty acid oxidase disorder, comprising administering to a patient a mitochondrial antiinflammatory composition in an amount effective to decrease inflammation in a patient.

3. The method of claim 1, for treating inflammation associated with rhabdomyolysis associated with a long-chain fatty acid oxidation disorder, comprising administering to a patient a mitochondrial antiinflammatory composition in an amount effective to treat rhabdomyolysis in a patient.

4. The method of claim 3, wherein the fatty acid oxidation disorder is rhabdomyolysis, and the treatment improves a symptom of rhabdomyolysis.

5. The method of claim 1, wherein the composition comprises D-Arg-2′6′-Dimethyltyrosine-Lys-Phe-NH₂, or an analog thereof, or a pharmaceutically-acceptable salt or ester thereof.

6. The method of claim 5, wherein the composition comprises D-Arg-2′6′-Dimethyltyrosine-Lys-Phe-NH₂, or a pharmaceutically-acceptable salt or ester thereof.

7. The method of claim 1, wherein the composition comprises 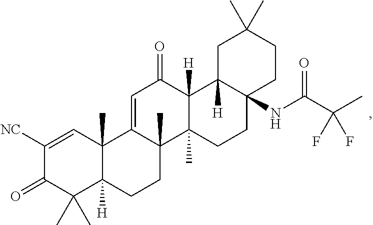
or an analog thereof, or a pharmaceutically-acceptable salt or ester thereof.

8. The method of claim 1, wherein the composition comprises: 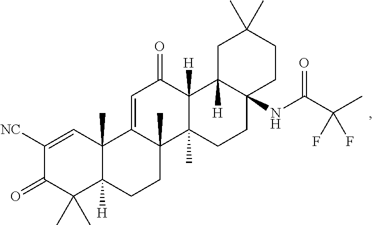
or a pharmaceutically-acceptable salt or ester thereof.

9. The method of claim 1, wherein the composition comprises one or more of triheptanoin or uridine triacetate.

10. A method of treating a fatty acid oxidation disorder in a patient, comprising administering to a patient an amount of a composition comprising: either D-Arg-2′6′-Dimethyltyrosine-Lys-Phe-NH₂, or a pharmaceutically-acceptable salt or ester thereof, or 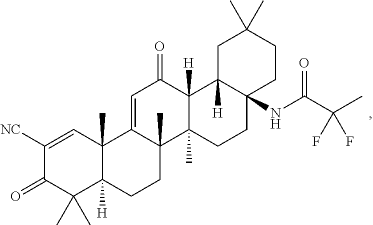
or a pharmaceutically-acceptable salt or ester thereof, effective to improve fatty acid metabolism and/or decrease inflammation in a patient.

11. (canceled)

12. A mitochondrial antiinflammatory composition, that optionally up-regulates fatty acid metabolism in a patient or stabilizes mitochondrial structure, in an amount effective to improve fatty acid oxidation function, improve mitochondrial respiratory chain function, increase mitochondrial stability, treat rhabdomyolysis, and/or decrease inflammation in a patient for use in treating a long-chain fatty acid oxidation disorder, treating inflammation associated with a long-chain fatty acid oxidase disorder, and/or treating inflammation associated with rhabdomyolysis associated with a long-chain fatty acid oxidation disorder.

13. The mitochondrial antiinflammatory composition of claim 12, wherein the fatty acid oxidation disorder is rhabdomyolysis, and a symptom of rhabdomyolysis is improved.

14. The mitochondrial antiinflammatory composition of claim 12, comprising D-Arg-2′6′-Dimethyltyrosine-Lys-Phe-NH₂, or an analog thereof, or a pharmaceutically-acceptable salt or ester thereof.

15. The mitochondrial antiinflammatory composition of claim 12, comprising 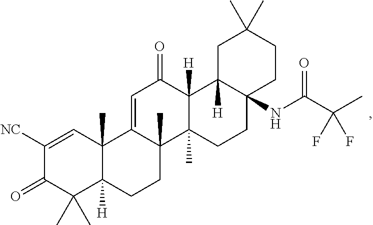
or an analog thereof, or a pharmaceutically-acceptable salt or ester thereof.
